(19) United States
(12) Patent Application Publication
(10) Pub. No.: US 2019/0261869 A1
(43) Pub. Date: Aug. 29, 2019

(54) SYSTEMS AND METHODS FOR MULTI-DISTANCE, MULTI-WAVELENGTH DIFFUSE CORRELATION SPECTROSCOPY

(71) Applicant: THE GENERAL HOSPITAL CORPORATION, Boston, MA (US)

(72) Inventors: Maria A. Franceschini, Boston, MA (US); Parisa Farzam, Boston, MA (US); Davide Tamborini, Boston, MA (US); David Boas, Boston, MA (US)

(21) Appl. No.: 16/349,405

(22) PCT Filed: Nov. 14, 2017
(86) PCT No.: PCT/US2017/061614

§ 371 Date: May 13, 2019

Related U.S. Application Data
(60) Provisional application No. 62/421,618, filed on Nov. 14, 2016.

(51) Int. Cl. A61B 5/026; A61B 5/00; A61B 5/1455

(52) U.S. Cl. CPC A61B 5/0261; A61B 5/0075; A61B 6/00; A61B 5/7203; A61B 5/14553

(57) ABSTRACT

The present disclosure provides systems and methods for multi-distance, multi-wavelength diffuse correlation spectroscopy (MD-MW DCS). The systems and methods can include two, three, or more different wavelengths and two, three, or more different source-detector distances. The dynamics of a target medium can be determined using detected signals at the different wavelengths and different source-detector distances.

### CROSS-REFERENCE TO RELATED APPLICATIONS

This application is related to, claims priority to, and incorporates herein by reference for all purposes U.S. Provisional Patent Application No. 62/421,618, filed Nov. 14, 2016.

### STATEMENT REGARDING FEDERALLY SPONSORED RESEARCH

This invention was made with government support under R01-GM116177-01A1 awarded by the National Institutes of Health. The government has certain rights in this invention.

### BACKGROUND

Diffuse correlation spectroscopy (DCS) is a method which can be used to measure blood flow non-invasively in buried tissues such as the brain from a sensor on the surface of the body. In the prior art, DCS measures microvascular blood flow index (BFi). DCS analysis requires prior information about absorption (μₐ) and reduced scattering (μₛ′) coefficients, so it is customary to deploy near-infrared spectroscopy (NIRS) in tandem with DCS in hybrid devices. These hybrid devices typically require separate light sources and detectors for the DCS and NIRS measurements.

There exists a need for new and improved systems and methods for measurement of fluid flow, and specifically, non-invasive measurement of blood flow. It would be beneficial if measurements that have traditionally required a DCS/NIRS hybrid system could be acquired with a single DCS system. It would also be beneficial to evaluate blood volume and hemoglobin oxygenation, which traditional DCS cannot achieve.

### SUMMARY

The present disclosure overcomes drawbacks of previous technologies by providing systems and methods for multi-distance, multi-wavelength diffuse correlation spectroscopy (MD-MW DCS).

In one aspect, the present disclosure provides a multi-distance, multi-wavelength diffuse correlation spectroscopy (MD-MW DCS) system. The system includes one or more DCS light sources, one or more DCS detectors, a memory, and a processor. The one or more DCS sources are configured to emit at least a first light having a first wavelength and a second light having a second wavelength. The one or more DCS light sources are configured to transmit the first light and the second light into a target medium. The first and second wavelength are different. The one or more DCS detectors are configured to receive at least a portion of the first light and at least a portion of the second light from the target medium. The DCS detector is configured to generate a DCS detector signal in response to receiving the at least a portion of the first light and the at least a portion of the second light. The memory stores one or more equations relating correlation to dynamics of scattering particles within the target medium. The processor is coupled to the one or more DCS detectors and the memory. The processor is configured to determine a dynamics of the target medium using the DCS detector signal and the one or more equations. The one or more DCS light sources and the one or more DCS detectors are configured to provide at least two different source-detector distances.

In another aspects, the present disclosure provides a method for making a multiple distance, multiple wavelength diffuse correlation spectroscopy (MD-MW DCS) measurement of scattering particle dynamics within a target medium. The method includes: a) coupling one or more DCS light sources and one or more DCS detectors to the target medium to provide at least two different source-detector distances, the one or more DCS light sources configured to emit at least a first light having a first wavelength and a second light having a second wavelength, the first wavelength and the second wavelength are different; b) transmitting the first light and the second light into the target medium; c) receiving at least a portion of the first light and at least a portion of the second light at the one or more DCS light detectors at both of the at least two different source-detector distances, thereby generating a DCS detector signal including photon arrival time information, wavelength information, and source-detector distance information; d) determining, using a processor and the DCS detector signal, a decay of an autocorrelation function over distance for at least the first wavelength and the second wavelength; e) determining, using the processor, the decay of the autocorrelation function over distance, and one or more equations relating the decay of the autocorrelation function over distance to optical properties and dynamics of the target medium, the dynamics of the target medium; and f) generating a report including the dynamics of the target medium.

In yet another aspect, the present disclosure provides a method. The method includes: a) coupling one or more DCS light sources and one or more DCS detectors to the target medium, the one or more DCS light sources configured to emit at least a first light having a first wavelength and a second light having a second wavelength, the first wavelength and the second wavelength are different, the one or more DCS light sources and the one or more DCS detectors are configured to provide at least two different source-detector distances; b) transmitting the first light and the second light into the target medium; c) receiving at least a portion of the first light and at least a portion of the second light at the one or more DCS light detectors at each of the two different source-detector distances, thereby generating a DCS detector signal including light intensity, autocorrelation, wavelength information, and source-detector distance information; d) determining, using (1) a processor, (2) the DCS detector signal, and (3) a global fitting method, (i) an absorption coefficient (μₐ), (ii) a reduced scattering coefficient (μₛ′), and (iii) a blood flow index (BFi); and e) generating a report including the absorption coefficient, the reduced scattering coefficient, or the blood flow index.

The foregoing and other advantages of the disclosure will appear from the following description. In the description, reference is made to the accompanying drawings which form a part hereof, and in which there is shown by way of illustration a preferred embodiment of the disclosure. Such embodiment does not necessarily represent the full scope of the disclosure, however, and reference is made therefore to the claims and herein for interpreting the scope of the disclosure.

### BRIEF DESCRIPTION OF THE DRAWINGS

The present disclosure will hereafter be described with reference to the accompanying drawings, wherein like reference numerals denote like elements.

FIG. 1 is a schematic of a system, in accordance with the present disclosure.

FIG. 2 is a schematic of a system, in accordance with the present disclosure.

FIG. 3 is a schematic of a light source control, in accordance with the present disclosure.

FIG. 4 is a schematic of a signal processor, in accordance with the present disclosure.

FIG. 5 is a schematic of a system, in accordance with the present disclosure.

FIG. 6 is an image of a probe, in accordance with the present disclosure.

FIG. 7 is a flowchart of a method, in accordance with the present disclosure.

FIG. 8 is a pair of plots of fitted blood flow index comparing the performance of three wavelengths versus one wavelength, as described in Example 1.

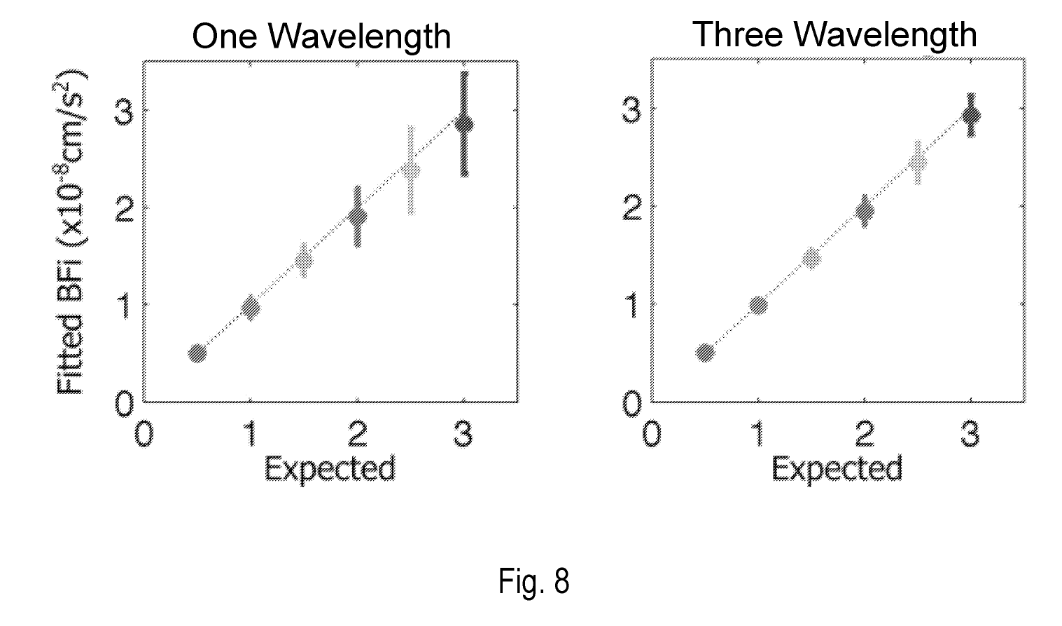

FIG. 9 is a normalized intensity autocorrelation function, as described in Example 2.

FIG. 10 is a plot of fitting error versus detector count rate, as described in Example 2.

FIG. 11 is a plot of the current versus time, as described in Example 2.

FIG. 12 is a plot of autocorrelation functions for four different wavelengths, as described in Example 2.

FIG. 13 is a plot of coherence and intensity versus time, as described in Example 2.

FIG. 14 is a series of plots showing absorption coefficient, reduced scattering coefficient, and the mean square displacement of the solution as a function of titration level for the absorption titration, as described in Example 2.

FIG. 15 is a series of plots showing absorption coefficient, reduced scattering coefficient, and the mean square displacement of the solution as a function of titration level for the scattering titration, as described in Example 2.

FIG. 16 is a series of plots showing absorption coefficient, reduced scattering coefficient, and the mean square displacement of the solution as a function of titration level for the dynamic titration, as described in Example 2.

### DETAILED DESCRIPTION

Before the present invention is described in further detail, it is to be understood that the invention is not limited to the particular embodiments described. It is also to be understood that the terminology used herein is for the purpose of describing particular embodiments only, and is not intended to be limiting. The scope of the present invention will be limited only by the claims. As used herein, the singular forms “a”, “an”, and “the” include plural embodiments unless the context clearly dictates otherwise.

It should be apparent to those skilled in the art that many additional modifications beside those already described are possible without departing from the inventive concepts. In interpreting this disclosure, all terms should be interpreted in the broadest possible manner consistent with the context. Variations of the term “comprising”, “including”, or “having” should be interpreted as referring to elements, components, or steps in a non-exclusive manner, so the referenced elements, components, or steps may be combined with other elements, components, or steps that are not expressly referenced. Embodiments referenced as “comprising”, “including”, or “having” certain elements are also contemplated as “consisting essentially of” and “consisting of” those elements, unless the context clearly dictates otherwise. It should be appreciated that aspects of the disclosure that are described with respect to a system are applicable to the methods, and vice versa, unless the context explicitly dictates otherwise.

Numeric ranges disclosed herein are inclusive, so recitation of a value of between 1 and 10 includes the values 1 and 10. Disclosure of multiple alternative ranges having different maximum and/or minimum values contemplates all combinations of the maximum and minimum values disclosed therein. For example, recitation of a value of between 1 and 10 or between 2 and 9 contemplates a value of between 1 and 9 or between 2 and 10 in addition to the positively recited values, unless explicitly stated to the contrary.

This disclosure provides systems and methods for multiple wavelength, multiple distance diffuse correlation spectroscopy (MD-MW DCS).

### Systems

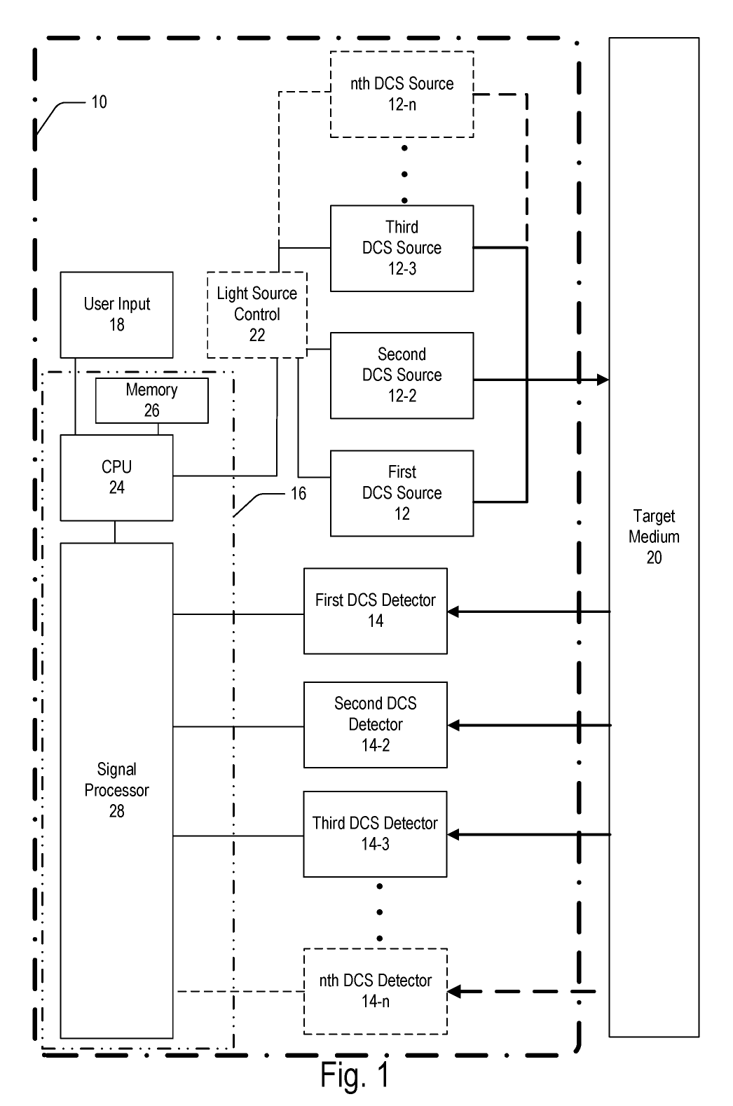

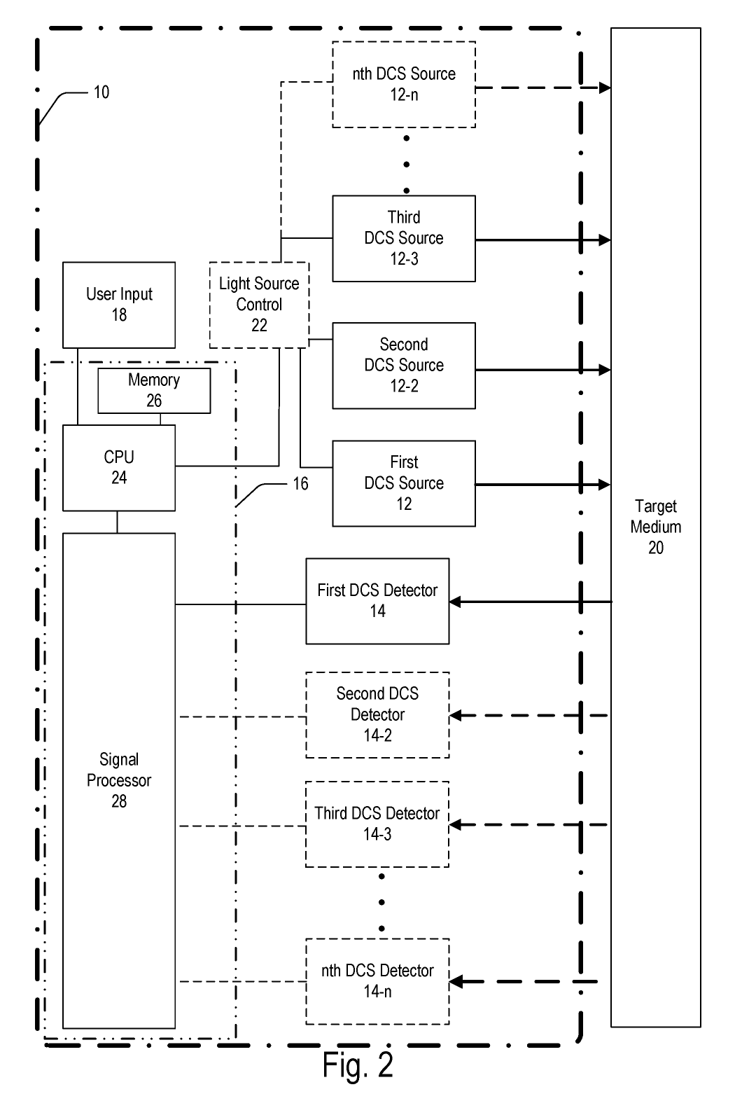

Referring to FIGS. 1 and 2, a system 10 suitable for executing the methods of the present disclosure is provided. The system 10 can include one or more DCS light sources 12, 12-2, 12-3, . . . , 12-n and one or more DCS detectors 14, 14-2, 14-3, . . . , 14-n. The system 10 can include a computer 16 in electronic communication with the one or more DCS light sources 12, 12-2, 12-3, . . . , 12-n and the one or more DCS detectors 14, 14-2, 14-3, . . . , 14-n. The system 10 can also include a user input 18 configured to provide an interface between a user and the computer 16 and/or other aspects of the system 10 (connections between the user input 18 and the other aspects are not illustrated, but can be appreciated by a person having ordinary skill in the art). The one or more DCS light sources 12, 12-2, 12-3, . . . , 12-n and the one or more DCS detectors 14, 14-2, 14-3, . . . , 14-n can be coupled to a target medium 20.

The first DCS light source 12 can be configured to transmit a first light into the target medium 20. The second DCS light source 12-2 can be configured to transmit a second light into the target medium 20. The third DCS light source 12-3 can be configured to transmit a third light into the target medium 20. The nth DCS light source 12-n can be configured to transmit an nth light into the target medium 20.

The one or more DCS light sources 12, 12-2, 12-3, . . . , 12-n can be a light source that is capable of emitting optical signals having the properties described elsewhere in the present disclosure. The one or more DCS light sources 12, 12-2, 12-3, . . . , 12-n can be a single-mode laser, a multi-mode laser, combinations thereof, and the like. The one or more DCS light sources 12, 12-2, 12-3, . . . , 12-n can be a diode laser, a solid-state laser, a fiber laser, a vertical cavity surface-emitting laser (VCSEL), a Fabry-Perot laser, a ridge laser, a ridge waveguide laser, a tapered laser, or other type of laser.

The one or more DCS light sources 12, 12-2, 12-3, . . . , 12-n can be configured to transmit light into the target medium 20 having a wavelength of between 400 nm and 1800 nm, including but not limited to, a wavelength of between 600 nm and 1000 nm, a wavelength of between 690 nm and 900 nm, a wavelength of between 450 nm and 750 nm, a wavelength of between 500 nm and 1250 nm, a wavelength of between 800 nm and 1350 nm, a wavelength of between 1000 nm and 1400 nm, or a wavelength of between 750 nm and 1450 nm. For aspects of the present disclosure using two or more different wavelengths, three or more different wavelengths, four, five, six, seven or more, or up to n different wavelengths, these ranges are also applicable. In some specific aspects using two different wavelengths, a first wavelength can be between of between 400 nm and 1800 nm, including but not limited to, a wavelength of between 600 nm and 1000 nm, a wavelength of between 690 nm and 900 nm, a wavelength of between 450 nm and 750 nm, a wavelength of between 500 nm and 1250 nm, a wavelength of between 800 nm and 1350 nm, a wavelength of between 1000 nm and 1400 nm, or a wavelength of between 750 nm and 1450 nm and a second wavelength can be between 400 nm and 1800 nm, including but not limited to, a wavelength of between 600 nm and 1000 nm, a wavelength of between 690 nm and 900 nm, a wavelength of between 450 nm and 750 nm, a wavelength of between 500 nm and 1250 nm, a wavelength of between 800 nm and 1350 nm, a wavelength of between 1000 nm and 1400 nm, or a wavelength of between 750 nm and 1450 nm. In some specific aspects using three different wavelengths, the first and second wavelengths can fall within the ranges described in the preceding sentence and a third wavelength can be between 400 nm and 1800 nm, including but not limited to, a wavelength of between 600 nm and 1000 nm, a wavelength of between 690 nm and 900 nm, a wavelength of between 450 nm and 750 nm, a wavelength of between 500 nm and 1250 nm, a wavelength of between 800 nm and 1350 nm, a wavelength of between 1000 nm and 1400 nm, or a wavelength of between 750 nm and 1450 nm.

In certain cases, the different wavelengths can be separated by between 10 nm and 500 nm, including but not limited to, between 15 nm and 400 nm, between 20 nm and 300 nm, between 25 nm and 250 nm, between 30 nm and 200 nm, between 40 nm and 100 nm, or between 50 nm and 75 nm. In certain cases, a first wavelength can be between 700 nm and 775 nm, a second wavelength can be between 775 nm and 825 nm, and a third wavelength can be between 825 nm and 900 nm. In certain specific cases, a first wavelength is 767 nm, a second wavelength is 80 nm, and a third wavelength is 852 nm.

The one or more DCS light sources 12, 12-2, 12-3, . . . , 12-n can be configured to transmit light into the target medium 20 having an average power of between 10 μW and 10 W, including but not limited to, an average power of between 100 μW and 200 mW, between 1 mW and 500 mW, or between 10 mW and 1 W. In some cases, the one or more DCS light sources 12, 12-2, 12-3, . . . , 12-n can be configured to provide the individual first light, second light, etc. at these powers or they can be configured to provide the first light, second light, etc. at a combined power within these ranges.

The one or more DCS light sources 12, 12-2, 12-3, . . . , 12-n can be configured to transmit light into the target medium 20 having a coherence length that is of the same order of magnitude as the path length distribution width of the light travelling through the target medium 20. The one or more DCS light sources 12, 12-2, 12-3, . . . , 12-n can be configured to transmit light into the target medium 20 having a coherence length of between 1.0 cm and 1 Km, including but not limited to, a coherence length of between 1.5 cm and 500 m, between 2.0 cm and 100 m, between 2.5 cm and 10 m, between 3.0 cm and 5 m, between 4.0 cm and 1 m, or between 5.0 cm and 50 cm.

Referring to FIGS. 1 and 2, in certain aspects, the system 10, 110 can further optionally include a fourth DCS light source, a fifth DCS light source, a sixth DCS light source, and so on, up to an nth DCS light source 12-n. Aspects of the present disclosure described with respect to one or more DCS light sources 12, 12-2, 12-3, . . . , 12-n are applicable to any number of DCS light sources 12, 12-2, 12-3, . . . , 12-n that are contained within the system 10, 110, so long as the wavelength requirements of the systems and methods are maintained. A person having ordinary skill in the art will appreciate that the number of DCS light sources is not intended to be limited in this disclosure, and the number exemplified by the illustrated aspects are specific only for ease of explanation and brevity. Similarly, the number of wavelengths can be increased beyond the illustrated and described aspects.

In certain aspects, the system 10 can further optionally include other light sources beyond the one or more DCS light sources 12, 12-2, 12-3, . . . , 12-n, which can collectively be referred to as additional light sources. These additional light sources can have similar properties to the one or more DCS light sources 12, 12-2, 12-3, . . . , 12-n or can have substantially different properties, and the different combinations and arrangements can have distinct advantages as described herein. In certain aspects, the additional light sources can be the sources listed with respect to the one or more DCS light sources 12, 12-2, 12-3, . . . , 12-n or can be a laser, a laser diode, an LED, a superluminescent diode, a broad area laser, a lamp, a white light source, and the like. In some cases, the additional light source or sources can be a NIRS light source or NIRS light sources.

Referring to FIG. 1, the system 10 is illustrated wherein the first DCS light source 12, the second DCS light source 12-2, the third DCS light source 12-3, and optional nth DCS light source 12-n are all coupled to the target medium 20 at a single transmission location. Referring to FIG. 2, a system 10 is illustrated wherein the first DCS light source 12, the second DCS light source 12-2, the third DCS light source 12-3, and optional nth DCS light source 12-n are all coupled to the target medium 20 at different transmission locations. In the case of FIG. 2, the first DCS detector 14, the second DCS detector 14-2, the third DCS detector 14-3, and the optional nth DCS detector 14-n can be coupled to the target medium 20 at the same or different detection locations.

The system 10 is configured to provide at least three source-detector distances. A first source-detector distance is the shortest source-detector distance, a second source-detector distance is longer than the first source-detector distance, and a third source-detector distance is longer than the second source-detector distance. The system 10 can be configured to provide four, five, six, and so on, up to n source-detector distances. The source-detector distances can be between 0.1 cm and 1 m, including but not limited to, between 0.2 cm and 50 cm, between 0.3 cm and 40 cm, between 0.4 cm and 30 cm, between 0.5 cm and 25 cm, between 0.6 cm and 20 cm, between 0.7 cm and 15 cm, between 0.8 cm and 10 cm, between 0.9 cm and 6.0 cm, between 1.0 cm and 5.0 cm, between 2.0 cm and 7.5 cm, between 2.5 cm and 12.5 cm, or between 3.0 cm and 8.0 cm. In certain aspects, a first source-detector distance can be between 0.1 cm and 2.0 cm. In certain aspects, a second source-detector distance can be between 1.0 cm and 3.0 cm. In certain aspects, a third source-detector distance can be between 1.0 cm and 5.0 cm. In certain aspects, a fourth source-detector distance can be between 1.0 cm and 6.0 cm. In certain aspects, a fifth source-detector distance can be between 1.0 cm and 6.0 cm. In one specific aspect, the first source-detector distance is 1.5 cm, the second source-detector distance is 2.0 cm, the third source-detector distance is 2.5 cm, and the fourth source-detector distance is 3.0 cm.

What follows is a non-limiting example of the use of the system 10 illustrated in FIGS. 1 and 2. In this aspect, one or more laser sources produce at least two distinct wavelengths of light. The at least two wavelengths are transmitted into a target medium.

The detected signals can be stored with a source-detector distance tag that identifies the source-detector distance for which signals were acquired. The detected signals can also be stored with a wavelength tag that identifies the wavelength at which the signals were acquired. In this aspect, the multi-distance DCS intensity measurements provide intensity decay over distance, which results in a slope that is proportional to the product μₐμₛ′. In certain aspects, the measurements of the decay of the autocorrelation function at three or more wavelengths can provide independent measurements to uniquely determine all parameters of interest for measuring fluid flow. These parameters can be used to estimate flow, hemoglobin concentrations and/or blood oxygenation, and result in improved accuracy, precision, and reduced variability with respect the prior art. The correlation functions can be autocorrelation functions calculated from individual detectors, autocorrelation functions calculated from multiple detectors, cross-correlation functions calculated between different detectors, or any combination thereof.

In certain aspects, the one or more DCS light sources 12, 12-2, 12-3, . . . , 12-n, the second DCS light source 12, 112-2, the third, fourth, fifth, up to nth DCS light source 12-n, 112-n, or any additional light sources can include one or more amplifiers to amplify the intensity of the emitted light.

In certain aspects, the additional light sources can have properties that are substantially similar to those described with respect to the DCS light source 12.

In certain aspects, the second DCS light source 12, 112-2, the third, fourth, up to nth DCS light source 12-n, 112-n, and/or additional DCS light sources can have properties that are substantially similar to those described with respect to the one or more DCS light sources 12, 12-2, 12-3, . . . , 12-n.

In some cases, the additional light sources or the additional DCS light sources can be configured to emit light that is substantially similar to the light emitted from the one or more DCS light sources 12, 12-2, 12-3, . . . , 12-n. In some cases, the additional light sources or the additional DCS light sources can be configured to emit light that is suitable for DCS, but having one or more different properties than the DCS light source.

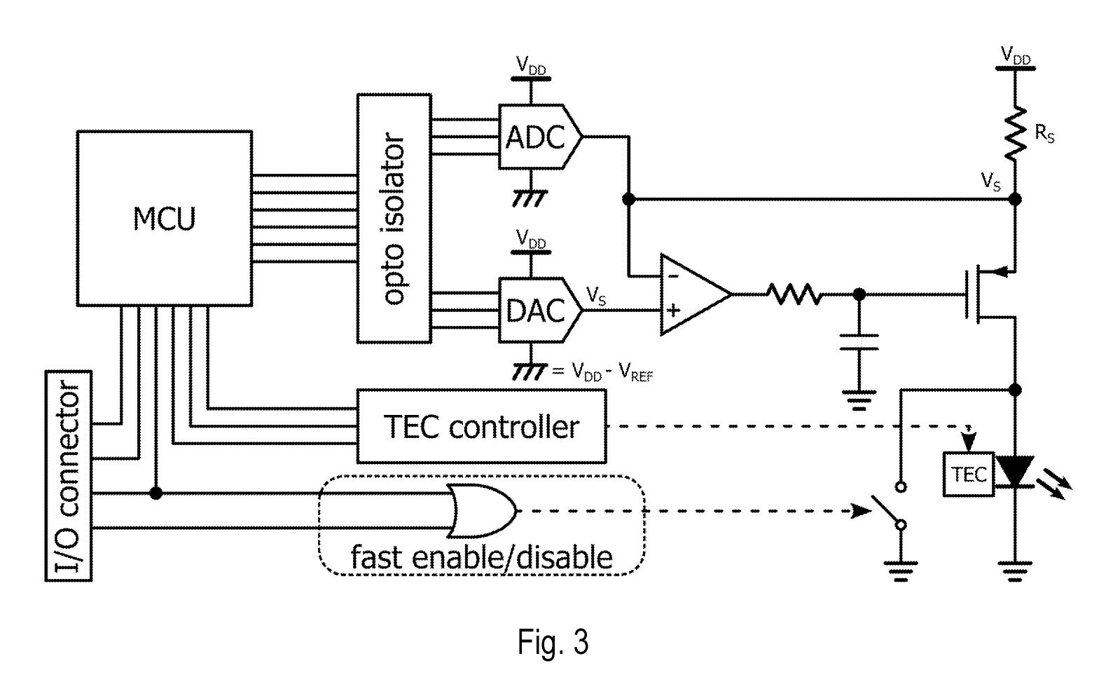

Referring to FIGS. 1 and 2, the DCS light sources 12, 12-2, 12-3, . . . , 12-n and additional light sources can be optionally be controlled by a light source control 22. The light source control can turn the DCS light sources 12, 12-2, 12-3, . . . , 12-n and additional light sources on and off in sequence. Referring to FIG. 3, one exemplary schematic for a light source control is illustrated.

In some cases, the light source control 22 can be configured to control the sequence of the source for time division multiplexing between different sources.

In certain aspects, the light source control 22 can be a component of the computer 16. In certain aspects, the light source control 22 can be a standalone component or multiple standalone components. One light source control 22 can control all or some of the various light sources or each of the various light sources can have its own light source control 22.

The one or more DCS detectors 14, 14-2, 14-3, . . . , 14-n can be a light detector that is capable of detecting optical signals having the properties described elsewhere in the present disclosure. In some cases, the one or more DCS detectors 14, 14-2, 14-3, . . . , 14-n can be an interferometric detector. The one or more DCS detectors 14, 14-2, 14-3, . . . , 14-n can be an avalanche photodiode detector, such as a single-photon avalanche photodiode detector, a photomultiplier tube, a Si, Ge, InGaAs, PbS, PbSe, or HgCdTe photodiode or PIN photodiode, phototransistors, MSM photodetectors, CCD and CMOS detector arrays, silicon photomultipliers, LCD, multi-pixel-photon-counters, spectrometers, and the like. In certain aspects, the one or more DCS detectors 14, 14-2, 14-3, . . . , 14-n can be enhanced to be sensitive to a specific wavelength of light. In certain aspects, the one or more DCS detectors 14, 14-2, 14-3, . . . , 14-n can function as a monitor photodiode. In certain aspects, the one or more DCS detectors 14, 14-2, 14-3, . . . , 14-n can be a multi-pixel photo-detector that can be utilized to obtain many parallel detection channels on a single detector. In certain aspects including such a detector, a smaller pixel size can increase the DCS contrast. The one or more DCS detectors 14, 14-2, 14-3, . . . , 14-n can be analog or photon counting.

The one or more DCS detectors 14, 14-2, 14-3, . . . , 14-n can provide a detector signal that can be analog, digital, photon-counting, or any combination thereof.

In some cases, the DCS detectors can be used to combine DCS with different modalities, such as near-infrared spectroscopy. However, as described elsewhere herein, a surprising result of the present disclosure is the ability to accurately estimate fluid dynamics without the need for near-infrared spectroscopy to measure properties of a target medium. That being said, nothing in the present disclosure is intended to limit the use of the systems and methods described herein with additional modalities.

In certain aspects, the system 10 can further optionally include additional detectors that can be utilized for conducting other forms of spectroscopic measurements. These additional detectors can have similar properties to the DCS detector 14 or can have substantially different properties, and the different combinations and arrangements can have distinct advantages as described herein. In some cases, the additional detector or additional detectors can be a NIRS detector or NIRS detectors.

In certain aspects, the one or more DCS detectors 14, 14-2, 14-3, . . . , 14-n, or any additional detectors can be configured to receive optical signals from a single location or from multiple locations. Any combination of DCS detection can be achieved with the same or different detectors, including various combinations of detectors.

The system 10 can optionally further include waveguides to couple the one or more DCS light sources 12, 12-2, 12-3, . . . , 12-n, the one or more DCS detectors 14, 14-2, 14-3, . . . , 14-n, the additional light sources, and/or the additional detectors to the target medium 20. The optional waveguides can be any waveguide suitable for delivering light having the properties described elsewhere herein. For example, the optical waveguides can be a fiber optic or a fiber optic bundle, a lens, a lens system, a hollow waveguide, a liquid waveguide, a photonic crystal, combinations thereof, and the like. The system 10 can also optionally include one or more lenses to couple the one or more DCS light sources 12, 12-2, 12-3, . . . , 12-n, the one or more DCS detectors 14, 14-2, 14-3, . . . , 14-n, the additional light sources, and/or the additional detectors to the target medium 20. The waveguides and lenses can be used together or separately.

The one or more DCS light sources 12, 12-2, 12-3, . . . , 12-n the one or more DCS detectors 14, 14-2, 14-3, . . . , 14-n, the additional light sources, and/or the additional detectors can be directly coupled to the target medium 20. In some cases, the coupling can be via direct contact with the target medium 20.

In certain aspects, the waveguides can be deployed in a probe, including as many waveguides as is practical. In certain aspects, the probe can be affixable to a head of a subject. In certain aspects, the probe can be configured to provide multiple distinct source-detector distances. In certain aspects, the waveguides can be deployed in a catheter.

The various DCS detectors 14, 14-2, 14-3, . . . , 14-n or additional detectors can have intervening optics and/or pin hole(s), holograms, and/or detector active area dimensions. The various DCS detectors 14, 14-2, 14-3, . . . , 14-n or additional detectors can be used singly, multiply, arrayed, or in any combination.

In certain aspects, the DCS detectors 14, 114, 14-2, 14-3, . . . , 14-n or additional detectors can have a small active area (i.e., 0.1 μm to 10 μm) to collect light from one or a few speckles, as can be required for DCS contrast, or can have a larger active area (i.e., 10 μm to 1 mm), which might not typically be associated with capabilities for DCS contrast. Combining different detectors with different performance for different modalities can have the advantage of improved overall performance and/or reduction in cost, weight, and/or power consumption. For example, the small active area required for DCS contrast can limit the maximum distance of the source-detector separation due to the decrease in transmission that is associated with a larger separation. On the other hand, time-resolved and continuous wave detection for non-DCS NIRS do not have this requirement, so detectors with different properties, including but not limited to a larger active area, a lower sensitivity, and the like, could be employed, using the same or different sources, or any combination of the above. Thus, a variety of source-detector separations can be utilized, thus enabling, for example, greater accuracy in determination of scattering and/or absorption coefficients than can be achieved using solely shorter separations. Some aspects have improved cost, weight, and/or power consumption. It should be appreciated that the specific aspects described are not intended to be limiting, and additional combinations of source or sources, detector or detectors, and distance or distances are possible.

The system 10 can also include various other optics that a person having ordinary skill in the art would appreciate as being useful for aiding the acquisition of optical measurement. The system 10 can include various lenses, filters, variable attenuators, polarizers, coupling optics, dielectric coatings, choppers (and corresponding lock-in amplification systems), pinholes, modulators, prisms, mirrors, fiber optic components (splitters/circulators/couplers), and the like.

In certain aspects, the one or more DCS detectors 14, 14-2, 14-3, . . . , 14-n can be configured to receive optical signals from multiple different waveguides. The multiple waveguides can be a part of an optical path that includes a filter.

The computer 16 can take the form of a general purpose computer, a tablet, a smart phone, or other computing devices that can be configured to control the measurement devices described herein, and which can execute a computer executable program that performs the simulations described herein. The computer 16 can include various components known to a person having ordinary skill in the art, such as a processor and/or a CPU 24, memory 26 of various types, interfaces, and the like. The computer 16 can be a single computing device or can be a plurality of computing devices operating in a coordinated fashion.

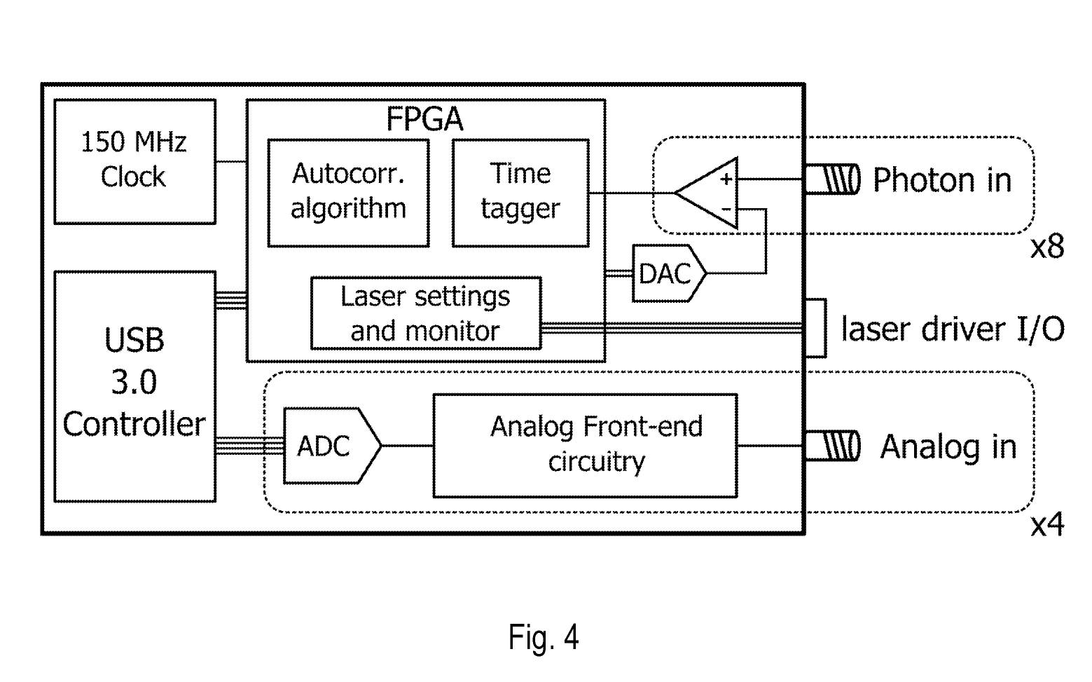

The computer 16 can include a signal processor 28 that is programmed to interpret the detected optical signals. For example, in some cases, the signal processor 28 can be configured to calculate autocorrelation and/or crosscorrelation functions. In some cases, the signal processor 28 can be configured to store photon arrival times and forward the arrival times for correlation processing. In some cases, the signal processor 28 can be configured to apply a correlation-diffusion equation. As non-limiting examples, the signal processor 28 can be implemented as a field-programmable gate array (FPGA), an application-specific integrated circuit (ASIC), a system on a chip (SOC), a microprocessor, a microcontroller, or the like. Referring to FIG. 4, a schematic of one specific signal processor 28 is shown.

In certain aspects, the signal processor 28 can be configured to extract measurement from the photon signals by a variety of means, including but not limited to, Fourier or other transform methods, heterodyning or homodyning methods, or a combination thereof, with examples including but not limited to, hardware-based extraction, software-based extraction, linear transforms, log transforms, multitau correlation, and combinations thereof.

A detector signal from one of the detectors can be multiplexed to individual processing paths, such as those discussed below, to be processed for DCS measurements. This multiplexing can afford efficiency in the processing.

The processor and/or CPU 24 can be configured to read and perform computer-executable instructions stored in the memory 26. The computer-executable instructions can include all or portions of the methods described herein.

The memory 26 can include one or more computer readable and/or writable media, and may include, for example, a magnetic disc (e.g., a hard disk), an optical disc (e.g., a DVD, a Blu-ray, a CD), a magneto-optical disk, semiconductor memory (e.g., a non-volatile memory card, flash memory, a solid state drive, SRAM, DRAM), an EPROM, an EEPROM, and the like. The memory can store the computer-executable instructions for all or portions of the methods described herein.

The user interface 18 can provide communication interfaces to input and output devices, which can include a keyboard, a display, a mouse, a printing device, a touch screen, a light pen, an optical storage device, a scanner, a microphone, a camera, a drive, a communication cable, or a network (wired or wireless). The interfaces can also provide communications interfaces to the one or more DCS light sources 12, 12-2, 12-3, . . . , 12-n, the one or more DCS detectors 14, 14-2, 14-3, . . . , 14-n, and other sources and/or detectors includes in the system 10 and/or used in the methods described herein.

The one or more DCS light sources 12, 12-2, 12-3, . . . , 12-n and the one or more DCS detectors 14, 14-2, 14-3, . . . , 14-n can be controlled by the computer 16. The computer 16 can have stored on it a computer executable program configured to execute such control. The computer 16 can direct the one or more DCS light sources 12, 12-2, 12-3, . . . , 12-n to emit optical signals that are configured to enter into the layered target medium in a fashion that allows the optical signals to interact with fluid flow in the target medium 20, including an inner region of the target medium 20. This interaction can allow the optical signals to acquire information related to the fluid flow in the inner region. The computer 16 can direct the one or more DCS detectors 14, 14-2, 14-3, . . . , 14-n to detect the optical signals that contain the acquired information.

In certain aspects, the system 10 can include an imaging modality or a layer thickness measuring modality for characterizing the target medium 20 and providing additional useful information. Examples of suitable imaging and/or layer thickness measuring modalities can include, but are not limited to, an ultrasound imaging system, a non-imaging ultrasound system configured to transmit and receive a reflected acoustic wave, an MRI imaging system, an x-ray imaging system, a computed tomography imaging system, a diffuse optical tomography imaging system, an optical layer thickness measurement system, combinations thereof, or the like. In other aspects, an ultrasound system could be configured to transmit an acoustic wave for depth-specific modulation of the light. Detecting this modulation in the DCS signal could further aid depth discrimination of the flow information.

In some aspects, the one or more DCS light sources 12, 12-2, 12-3, . . . , 12-n, the one or more DCS detectors 14, 14-2, 14-3, . . . , 14-n, the computer 16 of the system 10 and other components of the system 10 described herein, including additional DCS light sources and/or additional DCS detectors, can be contained in a single unit that is portable and suitable for point-of-care use. In some aspects, the single unit can be handheld. In some aspects, the computer 16 can be a handheld computing device and the remainder of the system 10 can be contained in a single unit that is portable and/or handheld. In some aspects, the system 10 can be contained in one or more handheld units. In some aspects, the system 10 or various components of the system 10 can be contained in a wearable device.

In some aspects, the one or more DCS light sources 12, 12-2, 12-3, . . . , 12-n, the one or more DCS detectors 14, 14-2, 14-3, . . . , 14-n, and the computer 16 of the system 10 and other components of the system 10 described herein, including additional DCS light sources and/or additional DCS detectors, can be contained in a table-top unit that is suitable for placement on a table-top and can be located appropriately for point-of-care use.

The system 10 can be powered by a power supply that is supplied electricity from a wall outlet or via one or more batteries, either rechargeable or replaceable.

It should be appreciated that various aspects of the system 10 that are illustrated as blocks are shown in this fashion for illustrative purposes, and those blocks can be multiple separate elements or can be combined into single monolithic elements.

Another advantage of the system 10 is that very small, lightweight detector fibers or solid state detectors can be used, and thus bendable probes can be used. In some aspects, the DCS system 10 can utilize the same small fibers or the same solid state components as a source and a detector, thereby reducing the number of fibers or electrical components required in a probe. Smaller probes can be desirable for vulnerable patients, such as infants, placement around surgical and/or wound sites, and for use with other measurement modalities, such as EEG, cranial bolts, and the like. Smaller probes are also advantageous for implantable, chronic, mobile, and/or wearable applications. Additional advantages can include reduced cost, weight, and/or power consumption.

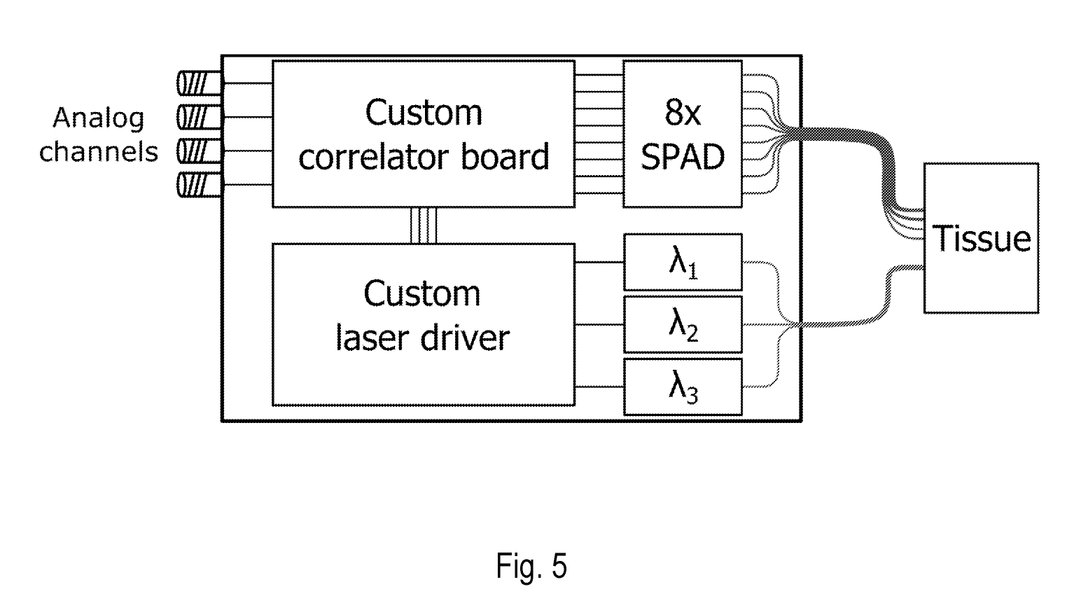

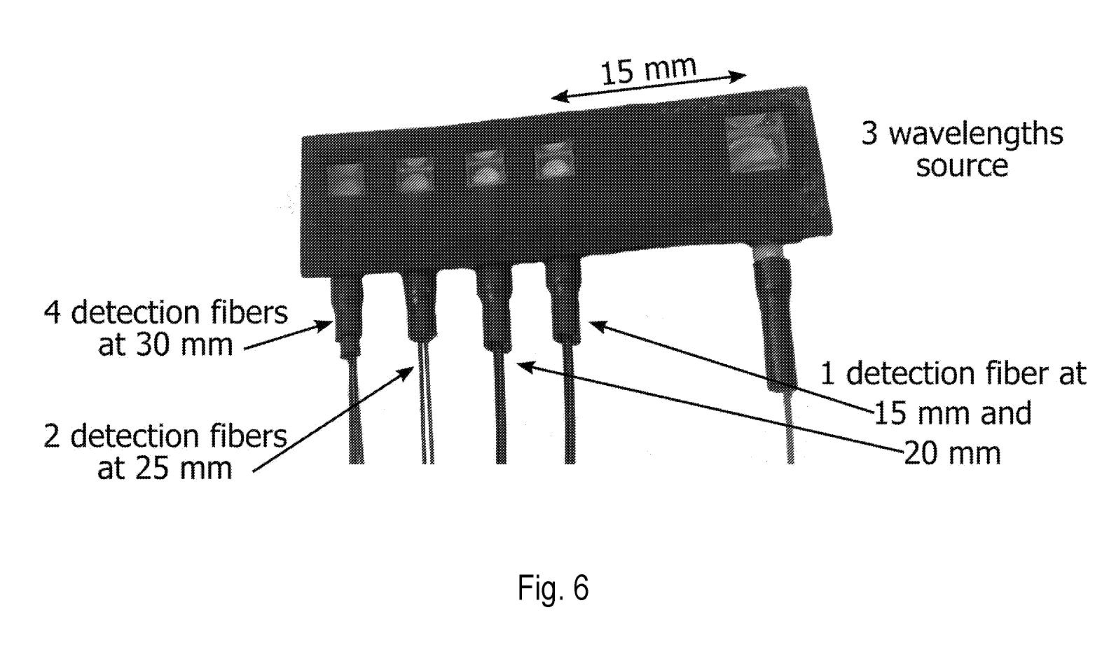

Referring to FIG. 5, one specific system arrangement is illustrated. Three light sources are configured to generate three different wavelengths of light and emit light having a long coherence length. Those lasers are controlled by a custom laser driver. The custom laser driver allows fast multiplexing of the three colors into a single transmission location within the probe. Referring to FIG. 6, an image of an exemplary probe is shown. The exemplary probe transmits three wavelengths of light from a single transmission location and collects light from four different source-detector distances, with one high-efficiency, single-photon avalanche photodiode detector located at a first, shortest source-detector distance, one high-efficiency, single-photon avalanche photodiode detector located at a second source-detector distance that is longer than the first source-detector distance, two high-efficiency, single-photon avalanche photodiode located a third source-detector distance that is longer than the second source-detector distance, and four high-efficiency, single-photon avalanche photodiodes located at a fourth source-detector distance that is longer than the third source-detector distance. A fifth, sixth, seventh, eighth, and so on, up to nth source-detector distance is also contemplated. The first source-detector distance illustrated is 15 mm, the second is 20 mm, the third is 25 mm, and the fourth is 30 mm, though other source-detector distances are contemplated. A custom FPGA-based correlator, such as the one illustrated schematically in FIG. 4, receives the detector signals and records an arrival time of each detected photon. A USB 3.0 interface is routed to a computer where software allows selection of a desired measurement repetition rate in post-processing, based on the desired signal-to-noise ratio. Fast acquisition rates allow measurement of pulsatile blood flow for better physiological noise filtering and quantification of additional parameters such as cerebrovascular reactivity (CVR) and intracranial pressure (ICP).

Aspects of the present disclosure discussed below with respect to the methods 100 are applicable to and can be incorporated in the systems 10 described herein. For clarity, if the methods below describe an aspect that a person having ordinary skill in the art would understand as implying the presence of structural features in the systems 10 described above, then this disclosure expressly contemplates the inclusion of those structural features. As a non-limiting example, if the methods below describe focusing light, then a person having ordinary skill in the art would understand that this implies the presence of a focusing lens or a structure that serves the purpose of a focusing lens, such as a concave curved mirror.

### Methods

This disclosure provides a method 100 for using the systems 10 described above, although the method 100 can optionally be used with other systems not described herein.

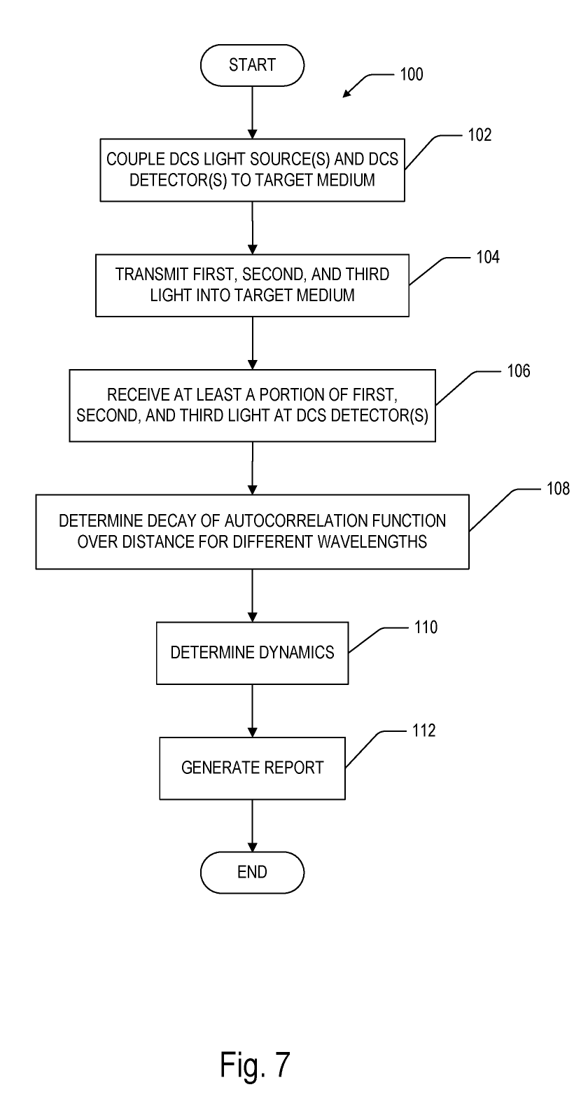

Referring to FIG. 7, the present disclosure provides a method 100 for making a multi-distance, multi-wavelength DCS measurement within a target medium. At process block 102, the method 100 includes coupling one or more DCS light sources and one or more DCS detectors to the target medium. The one or more DCS light sources are configured to at least two different wavelengths of light. The one or more DCS light sources and the one or more DCS detectors are configured to provide at least two different source-detector distances. At process block 104, the method 100 includes transmitting first and second light having the at least two different wavelengths of light into the target medium. At process block 106, the method 100 includes receiving at least a portion of the first and second light at the one or more DCS detectors, thereby generating a DCS detector signal. The DCS detector signal includes photon arrival time information, wavelength information, and source-detector distance information. The receiving is performed at each of the at least two different source-detector distances. At process block 108, the method 100 includes determining a decay of an autocorrelation function over distance for at least the first and second wavelength, using a processor and the DCS detector signal. At process block 110, the method 100 includes determining a dynamics of the target medium. The determining of process block 110 can use the processor, the decay of the autocorrelation function over distance, and one or more equations relating the decay of the autocorrelation function over distance to optical properties and dynamics of the target medium. At process block 112, the method 400 includes generating a report including the dynamics of the target medium.

In some cases, process blocks 102, 104, and 106 can be repeated with different source-detector distances. In some cases, process blocks 102, 104, and 106 can be conducted for different source-detector distances simultaneously. In some cases, process blocks 102, 104, and 106 can include a third light having a third different wavelength, a fourth light having a fourth different wavelength, and so on, up to an nth light having an nth different wavelength. In some cases, process blocks 102, 104, and 106 can include a third different source-detector distance, a fourth different source-detector distance, a fifth different source-detector distance, and so on, up to an nth different source-detector distance.

The determining of process blocks 108 and 110 can utilize the different distances. The determining of process blocks 108 and 110 can include calculating using one or more of the equations or concepts described herein. The determining of process blocks 108 and 110 can include fitting data in ways known to those having ordinary skill in the art. The determining of process blocks 108 and 110 can be executed on a processor or CPU 24.

The generating a report of process block 112 can include generating a printed report, displaying results on a screen, transmitting results to a computer database, or another means of reporting the mathematically modeled fluid flow, as would be apparent to a person having ordinary skill in the art. The method is not intended to be limited to a specific report generation.

In certain aspects, the dynamics that are determined by the methods described herein can be fluid flow, shear flow, diffusional properties, motion, association, dis-association, aggregation, dis-aggregation, and/or rotational dynamics of the optical scattering particles within the target medium, and the like.

In certain aspects, dynamics and/or fluid flow can be determined from by calculating the correlation function from the path length distribution for the given coherence length of the light and/or path length of the reference optical path. Other aspects can utilize other means of measuring dynamics and/or fluid flow, including but not limited to, power spectrum analysis, moment analysis, and the like. The analysis can be performed singly, and/or independently or globally across multiple groups, or combinations thereof. The analysis can be performed by components of the system 10 described above that a person having ordinary skill in the art would appreciate as being capable of the analysis.

In certain aspects, the methods described herein can utilize measurement at two, three, four, five, six, or more, up ton source-detector distances. Use of multiple source-detector distances can provide better discrimination between various different depths of measurement, such as between cerebral and extra-cerebral measurements, and can provide increased accuracy for the estimation of the properties of the medium and corresponding flow determinations.

In certain aspect, the methods described herein can combine DCS with CW and time-domain or frequency-domain NIRS. Again, one of the surprising advantages of the present disclosure is that the need for NIRS to determine properties of the target medium is no longer required. However, the systems and methods described herein can still be used with NIRS without deviating from the present disclosure.

In certain aspects, the methods described herein can measure properties of the target medium 20 in a baseline state, in a state of spontaneous change, in an evoked change, or a combination thereof. Comparing the measurement of a property following an evoked change with a measurement at a baseline state can provide information regarding the evoked change.

In certain aspects, the methods described herein can utilize detected signals from a single site or multiple sites.

In certain aspects, the correlation described herein can be normalized or unnormalized.

In certain aspects, the methods described herein can measure the optical properties of the target medium 20 at the same wavelength and in the same location. The measured properties can be used to reduce intra- and inter-subject variability due to anatomy and physiology.

Calculations, separation, and/or discrimination in the methods described herein can be performed in real-time, near real-time, post-processing, or a combination thereof. These operations can be performed continuously, quasi-continuously, and/or continually, or periodically, and/or intermittently or in batches, or any combination thereof. Alerts, alarms, and/or reports can be generated in response to the results. The alerts, alarms, reports, and/or results can be displayed locally and/or remotely transmitted.

The target medium 20 can include an inner region and a superficial layer. The superficial layer can include one, two, three, four, five, six, or more distinct layers. In some aspects, the superficial layer can include two, three, or four distinct layers.

The superficial layer can include a skull of a subject, a scalp of a subject, a fluid layer between the skull and a cerebral region of a subject, or a combination thereof. The inner region can include a cerebral region of a subject.

The fluid can be blood, water, cerebro spinal fluid (CSF), lymph, urine, and the like. The fluid flow can be blood flow, water flow, CSF flow, lymph flow, urine flow, and the like.

In certain aspects, the target medium 20 can be an industrial fluid of interest. In certain aspects, the target medium 20 can be tissue, including but not limited to, mammalian tissue, avian tissue, fish tissue, reptile tissue, amphibian tissue, and the like. In certain aspects, the target medium 20 can be human tissue.

Aspects of the present disclosure discussed above with respect to the systems 10 are applicable to and can be incorporated in the methods 100 described herein. For clarity, if the systems above describe a structural feature that a person having ordinary skill in the art would understand as implying the presence of a method step or feature in the methods described above, then this disclosure expressly contemplates the inclusion of those method steps or features. As a non-limiting example, if the systems above describe a focusing lens that receives a collimated light beam, then a person having ordinary skill in the art would understand that this implies the presence of a method step or feature involving focusing of light.

### Computational Considerations

DCS measures the temporal speckle fluctuations due to the moving scatterers in tissue (red blood cells), which in turn could be used to estimate an index of blood flow in the microvasculature (see, D. A. Boas and A. G. Yodh, “Spatially varying dynamical properties of turbid media probed with diffusing temporal light correlation,” Journal of the Optical Society of America A, vol. 14, no. 1, pp. 192-215 (1997); D. A. Boas, L. E. Campbell and A. G. Yodh, “Scattering and Imaging with Diffusing Temporal Field Correlations,” Physical Review Letters, vol. 75, no 9, pp. 1855-1858 (1995); and D. Boas, S. Sakažic, J. Selb, P. Farzam, M. Franceschini and S. Carp, “Establishing the diffuse correlation spectroscopy signal relationship with blood flow,” Neurophotonics, vol. 3, no. 3, p. 031412 (2016)). The dynamic motion of the medium can be determined by measurement of the autocorrelation function, as faster motion of the scatterers is indicated by faster speckle fluctuations (i.e., more rapid decay of the autocorrelation function). The Green's function solution of the correlation diffusion equation for semi-infinite boundary conditions (see, T. Durduran, R. Choe, W. B. Baker and A. G. Yodh, “Diffuse optics for tissue monitoring and tomography”, Reports on Progress in Physics, vol. 73, no 7, p. 076701 (2010)) is:

$$\begin{matrix}{G}_{1} ( ρ , τ , λ ) = \frac{3 {μ}_{s}^{′} ( λ )}{4 π} [ \frac{{e}^{- K ( τ , λ ) {r}_{1} ( ρ , λ )}}{{r}_{1} ( ρ , λ )} - \frac{{e}^{- K ( τ , λ ) {r}_{b} ( ρ , λ )}}{{r}_{b} ( ρ , λ )} ] \\ where & ( 1 ) \\ {r}_{1} ( ρ , λ ) = \sqrt{1 / {{μ}_{s}^{′} ( λ )}^{2} + {ρ}^{2}} , & ( 2 ) \\ {r}_{b} ( ρ , λ ) = \sqrt{{( 2 {z}_{b} + \frac{1}{{μ}_{s}^{′} ( λ )} )}^{2} + {ρ}^{2}} , & ( 3 ) \\ K ( τ , λ ) = \sqrt{3 {μ}_{a} ( λ ) {μ}_{s}^{′} ( λ ) + 6 {{μ}_{s}^{′} ( λ )}^{2} {k}_{0}^{2} ( λ ) {BF}_{i} τ} , \\ {z}_{b} = 2 / {μ}_{s}^{′} ( 1 + {R}_{eff} ) / ( 1 - {R}_{eff} ) , & ( 4 )\end{matrix}$$

R_{eff} is the effective reflection coefficient to account for the index mismatch between tissue and air, k₀=2π/λ is the wave-number of light in the medium, corrected λ is the light wavelength, τ is the delay time, ρ is the source-detector separation, BFᵢ is the quantitative blood flow index, μₐ and μₛ′ are respectively the absorption and reduced scattering coefficients. The blood flow index is historically described as the probability of a dynamic scattering event (i.e., scattering from a red blood cell) times the mean square displacement of the dynamic scatterers (i.e., red blood cells) (see, T. Durduran and A. G. Yodh, “Diffuse correlation spectroscopy for non-invasive, micro-vascular cerebral blood flow measurement,” Neuroimage, vol. 85, pt 1, pp. 51-63 (2013); and Boas 1997). We recently showed that it can be explicitly related to the absolute blood flow as given by Eq. 15 in Boas 2016, where the probability of scattering from a red blood cell has a potential wavelength dependence given by the blood reduced scattering coefficient divided by the tissue reduced scattering coefficient. This potential wavelength dependence is negligible as both the blood and tissue scattering coefficients vary with wavelength in a similar way. We thus ignore this potential wavelength dependence here, but will discuss its potential impact in the discussion section. Then, in our homogeneous dynamic phantom measurements (described below) the probability of a dynamic scattering event is equal to 1 and is not wavelength dependent.

DCS measures the normalized intensity autocorrelation function (g₂), while the correlation diffusion equation applies to the electric field autocorrelation function. To fit the theory to the experimental data, the normalized intensity autocorrelation function must be related to the normalized electric field temporal autocorrelation (g₁) through the Siegert relation (see, P. A. Lemieux and D. J. Durian “Investigating non-gaussian scattering processes by using nth-order intensity correlation functions,” Journal of the Optical Society of America A, vol. 16, pp. 1651-64 (1999)):

g₂(τ,ρ,λ)=1+βg₁(τ,ρ,λ)², (5)

where β is a constant determined primarily by the optics of the experiment and it is related to the number of modes in the detected light. In most DCS experiments, employing coherent, non-polarized sources and single mode detector fibers, β is approximately 0.5 (see, L. He, Y. Lin, Y. Shang, B. J. Shelton and G. Yu, “Using optical fibers with different modes to improve the signal-to-noise ratio of diffuse correlation spectroscopy flow-oximeter measurements,” Journal of Biomedical Optics, vol. 18, no. 3, p. 37001 (2013)).

We aim to decouple the contribution of static (absorption and scattering) and dynamic (flow) properties of the tissue at large separations, which enables us to simultaneously estimate BE, hemoglobin oxygenation (SO₂) and oxygenated and deoxygenated hemoglobin concentrations (HbO and HbR, respectively). To this end, assuming a homogeneous medium, we use the DCS information (light intensity and g₂ curves) obtained from multiple wavelengths at multiple source-detector separations to fit for the desired parameters (μₐ, μₛ′ and BF).

By measuring the light intensity at each separation and wavelength, I(ρ, λ), and by calibrating sources and detectors, for each wavelength, we obtain the effective attenuation coefficient:

μ_{eff}(λ)=\sqrt{3μₛ′(λ)·μₐ(λ)}, (6)

since μ_{eff}(λ) is given by the slope of the simplified solution of the diffusion equation (see, Durduran 2010) versus distance:

ln(ρ²I(ρ,λ))=−μ_{eff}(λ)ρ+I₀(ρ=0,λ). (7)

To aid convergence of the fitting algorithm, we apply additional constrains. First BE is constant over wavelengths, since flow is a mechanical property of the medium and does not depend on the wavelength used to perform the measurement. Then we take into account the wavelength dependence of μₛ′ and μₐ. The reduced scattering coefficient in tissue follows an empirical power law relationship (see, H. J. van Staveren, C. J. M. Moes, J. van Marie, S. A. Prahl and M. J. C. van Gernert, “Light scattering in lntralipid-10% in the wavelength range of 400-1100 nm,” Applied Optics, vol. 30, no. 31, p. 4507 (1991); J. R. Mourant, J. P. Freyer, A. H. Hielscher, A. A. Eick, D. Shen and T. M. Johnson, “Mechanisms of light scattering from biological cells relevant to noninvasive optical-tissue diagnostics,” Applied Optics, vol. 37, no. 16, pp. 3586-3593 (1998); X. Wang, B. W. Pogue, S. Jiang, X. Song, K. D. Paulsen, C. Kogel, S. P. Poplack and W. A. Wells, “Approximation of Mie scattering parameters in near-infrared tomography of normal breast tissue in vivo,” Journal of Biomedical Optics, vol. 10, no. 5, p. 051704 (2005); S. L. Jacques and B. W. Pogue, “Tutorial on diffuse light transport”, Journal of Biomedical Optics, vol. 13, no. 4, p. 041302 (2008); and S. L. Jacques, “Optical Properties of Biological Tissues: A Review,” Physics in Medicine and Biology, vol. 58, no. 11 (2013)):

μₛ′(λ)=aλ^{-b}, (8)

where a is the scaling factor and b is the scattering power, both independent from λ. The absorption coefficient in tissues linearly depends on the hemoglobin concentrations as:

μₐ(λ)=∈_{HbO}(λ)·HbO+∈_{HbR}(λ)·HbR+p_{H}_{₂}_{O}·μ_{a(H}_{₂}_{O)}(λ), (9)

where ∈(λ) are the wavelength-dependent oxy and deoxy-hemoglobin extinction coefficients, obtained from the literature (see, Prahl S. Optical absorption of hemoglobin. http://omlc.ogi.edu/spectra. Accessed 2013), and p_{H}_{₂}_{O} is the assumed percent of water in tissue (see, D. R. White, H. Q. Woodard and S. M Hammond “Average soft-tissue and bone models for use in radiation dosimetry,” The British Institute of Radiology, vol. 60, no. 717, pp. 907-913 (1987); and D. R. White, E. M. Widdowson, H. Q. Woodard and J. W. Dickerson, “The composition of body tissues. (II) Fetus to young adult,” The British Institute of Radiology, vol. 64, no. 758, pp. 149-159 (1990)).

The MD-MW DCS global fitting is performed to fit experimental data over λ, τ, ρ to minimize the cost function (χ²) to fit for BE, a, b, HbO and HbR, that are independent from wavelength and distance:

$$\begin{matrix}{χ}^{2} = {\sum }_{k = 1}^{{N}_{λ}} \frac{1}{{N}_{τ}} ( {\sum }_{j = 1}^{{N}_{ρ}} \vert {\sum }_{m = 1}^{{N}_{τ}} \\ ( {g}_{1}^{theory} ( {ρ}_{j} , {τ}_{m} , {λ}_{k} , {BF}_{i} , a , b , HbO , HbR ) - \\ { {g}_{1}^{measured} ( {ρ}_{j} , {τ}_{m} , {λ}_{k} , {BF}_{i} , a , b , HbO , HbR ) )}^{2} \vert \\ γ \vert {μ}_{eff}^{theory} ( {λ}_{k} , a , b , HbO , HbR ) - \\ {μ}_{eff}^{measured} ( {λ}_{k} , a , b , HbO , HbR ) \vert ) ,\end{matrix} \qquad ( 10 )$$

where γ is a scaling factor that changes the weight of μ_{eff} in the fitting procedure. When γ=0, the fitting discards the information from intensity and the fitted μ_{eff} does not necessarily match the measured μ_{eff}. By increasing γ towards higher values, we are enforcing the fitted μ_{eff} to match the measured one. The optimal γ depends on the relative noise levels in the intensity and autocorrelation function data. For our system and experiments γ between 0.05 and 0.5 provides the best estimates of the optical properties and BFᵢ. We used γ=0.3 for the phantom experiment results presented below.

Finally, from the five fitted parameters we can calculate μₛ′ and μₐ at each wavelength using Eqs. 8 and 9, as well as total hemoglobin concentration (HbT=HbO+HbR) and oxygenation (SO₂=HbO/HbT).

### EXAMPLES

### Example 1

Numerical simulations for multi-distance measurements at a single wavelength were compared with simulations for multi-distance measurements at three wavelengths. The results are shown in FIG. 5.

### Example 2

We validated the MD-MW DCS approach with measurements in tissue like phantoms.

We used liquid mixtures of water, Intralipid and black India ink to increase the absorption or scattering of the solution at regular increments. For the absorption titrations, we mixed 40 ml of 20% Intralipid suspension in 1600 ml of water to achieve a scattering coefficient of 5.5 cm⁻¹ at 808 nm. There was initially no absorption (beyond the water itself), and progressive amounts of diluted India ink were added to increase optical absorption to 450% of the initial value. For the scattering titrations, we started by mixing 20% Intralipid with water and India ink to achieve an absorption of about 0.03 cm⁻¹ at 808 nm. Additional amounts (8 ml) of concentrated Intralipid were added until scattering increased by 150% of the initial value. The liquid mixture was stirred using a magnetic stirrer after every ink step, and it was allowed to come to rest before taking a measurement, about 2 minutes (monitored by following the return of the DCS autocorrelation function to a stable value). To simulate blood flow changes, we used the same stirrer and left it on at different levels during the measurements to increase the dynamics of the liquid phantom. For simplicity in this manuscript we call BFᵢ the mean square displacement of the scattering particles within the solution. For these measurements we used a silicone oil based solution, much more viscous than the water, and an Intralipid/India ink solution was used for the absorption and scattering titrations. This silicon based solution allowed us to perform measurements at 9 stirring levels and increase BFᵢ by 1400% from the initial value.

All measurements were done at a constant temperature of 20° C. (changes in temperature affect the Brownian motion of the solution). At each titration step, measurements were done for 20 second per wavelength with the MD-MW DCS system. In addition to the measurements at the three MD-MW DCS wavelengths (767, 808 and 852 nm) we used an additional laser at 785 nm (CL-2000 Diode Pumped Crystal, by CrystaLaser) to check potential improvements using 4 wavelengths. The recovered optical properties were compared to the optical properties simultaneously measured with a commercial frequency-domain near infrared spectroscopy (FDNIRS) system (MetaOx, ISS Inc.) (see, S. Fantini, M. A. Franceschini, J. S. Maier, S. A. Walker, B. B. Barbieri and E. Gratton, “Frequency-domain multichannel optical detector for noninvasive tissue spectroscopy and oximetry,” Optical Engineering, vol. 34, no. 1, pp. 32-42 (1995)). The estimated BFi using the MD-MW DCS method was compared with the BFi calculated using the FDNIRS optical properties at corresponding wavelengths. Interpolations based on the phantom spectral wavelength dependence were used for the optical properties at 850 nm. The FDNIRS multi-distance method achieved with a combination of different detectors requires calibration to correct for differences in coupling, gain and fibers transmission of the detectors, and it is done in a solid phantom of known optical properties (see, S. A. Carp, P. Farzam, N. Redes, D. M. Hueber and M. A. Franceschini, “Combined multi-distance frequency domain and diffuse correlation spectroscopy system with simultaneous data acquisition and real-time analysis,” Biomedical Optics Express, vol. 8, no. 9, pp. 3993-4006 (2017)). Our DCS MD-MW method requires a similar calibration of the light intensity to estimate μ_{eff}, but because of speckle noise in a solid phantom, it is preferable to use a liquid phantom as a reference in which the speckle intensity rapidly fluctuates and thus we measure the true average intensity with a short temporal average. In fact, DCS measures a single speckle and must average over longer time than the speckle fluctuation time to estimate the average intensity, while NIRS uses larger detection fibers to average over thousands of speckles and thus measures the average intensity directly. Therefore, DCS intensity was calibrated in the liquid phantom on the first titration, by using the optical properties recovered by FDNIRS. We verified the consistency of the data when calibrating on the last titration. The DCS and FDNIRS optical probes were immerged in the solution on opposite sides of the beaker container, far enough to avoid cross talk and allow for simultaneous acquisition. Both probes had the same source detector separations (15, 20, 25 and 30 mm).

A block diagram of the MD-MW DCS system is shown in FIG. 5: it makes use of three long-coherence lasers at three different wavelengths in the near-infrared spectral region and eight single-photon detectors to collect light at multiple distances. The lasers are driven by custom circuitry and the output light is delivered to the tissue through fiber optics to a source location in the optical probe. The light propagated through the tissue is collected by single mode fibers located at different distances in the optical probe and is delivered to the single-photon detectors. The detector's outputs are sent to a custom-built FPGA-based Correlator board. Four analogue channels are used to record physiological signals. Finally, a USB 3.0 controller is used to transfer DCS and auxiliary data to a remote PC.

Standard DCS systems use one long-coherence length laser operated in CW mode, and continuously detects the light at the detector. Multiple detectors are typically used in the same location to average autocorrelation functions and improve SNR. In our approach, we need to use three or more lasers at different wavelengths and multiple detectors at different distances. To minimize costs and avoid crosstalk between wavelengths, we use a temporal multiplexing approach of turning laser sources on and off in sequence. This approach is intrinsically crosstalk free, minimizes the number of detectors needed by employing the same detector for the different colors, and minimize light losses since it does not require the use of filters to block different wavelengths. The only drawback is the longer measurement time increased by a factor proportional to the number of wavelengths measured. For this approach, we designed and built a laser driver able to provide a stable current to the lasers and to rapidly multiplex the three colors.

We also developed a correlator board that provides the multiplexing signals and performs autocorrelation functions synchronized to each wavelength.

To implement the temporal multiplexing approach for providing multi-wavelengths to the tissue, it is necessary to be able to quickly enable/disable the DCS light sources. We selected the monolithic distributed Bragg reflector (DBR) lasers at 767, 808, 852 nm (PHxxxDBR series, by Photodigm Inc.). These lasers have a coherence length of tens of meters, a maximum optical power higher than 100 mW when operated in CW, and a sub-nanosecond turn on/off time that allows also pulsed mode operation (see, J. Sutin, B. Zimmerman, D. Tyulmankov, D. Tamborini, K. C. Wu, J. Selb, A. Gulinatti, I. Rech, A. Tosi, D. A. Boas and M. A. Franceschini, “Time-domain diffuse correlation spectroscopy,” Optica, vol. 3, pp. 1006-1013 (2016)). Each laser is packaged with a thermoelectric cooler (TEC) to keep a stable temperature for improving the laser coherence length.

FIG. 3 shows a schematic of the custom-built laser driver based on an ultra-stable, low-noise current generator capable of providing to the laser a current configurable between 0 and 500 mA. The driver core is basically a standard current generator (see, P. Horowitz and W. Hill, “Operational Amplifier,” in The Art of Electronics, 3rd ed. Cambridge University Press, Cambridge, England (2015)), composed of a PMOS transistor able to provide the current set by the sense resistor (R_{S}) through a digital set point (V_{S}) and an operational amplifier to provide a stable, negative feedback. In order to achieve a long-coherence length, we carefully designed the current generator selecting low-noise, low-drift components, filtering the supply rails and minimizing the set point disturbances (see, C. J. Erickson, M. Van Zijll, G. Doermann and D. S. Durfee, “An ultrahigh stability, low-noise laser current driver with digital control,” Review of Scientific Instruments, vol. 79, p. 073107 (2008)).

In particular, the 5Ω sense resistor (Z Series Vishay Foil Resistors, by Vishay) has a 0.05% tolerance and a 0.05 ppm/° C. drift, and the operational amplifier (AD8675, by Analog Devices Inc.) has a 0.2 μV/° C. drift and only a 2.8 nV/√Hz noise spectral density. The digital set point is provided by a 16-bit DAC (Digital-to-Analog Converter) with a 0.05 ppm/° C. drift and a 11.8 nV/√Hz noise spectral density (AD5541A, by Analog Devices Inc.). The DAC is powered between the laser supply (V_{DD}) and V_{DD}-V_{REF}, where V_{REF} is a 2.5 V precise voltage reference (VRE3025JS, by Apex Microtechnology Inc.), in order to minimize the effect of noise and disturbances on V_{DD}. When the set point is configured as V_{S}=V_{DD}, the voltage drop over R_{S} (given by V_{DD}−V_{S}) is 0, resulting in no current flowing into the laser, while setting V_{S}=V_{DD}−V_{REF}, the voltage drop over R_{S} is V_{REF}, resulting in the maximum current of V_{REF}/R_{S}=500 mA flowing into the laser. The current generator is also isolated and its power supplies are properly filtered to further minimize noise and disturbances.

A microcontroller unit (MCU) (ATMEGA2561, by Atmel Corp.) handles the current settings, the temperature of the laser through a TEC controller (1MD03-024-04/1, by RMT Ltd), and the communication to the system. There is also a precise, 18-bit ADC (Analog-to-Digital Converter) (AD7690, by Analog Devices Inc.) to monitor the current generator and to allow the implementation of a digital control loop. A fast enable/disable logic allows the MCU or an external signal to turn on/off the laser in less than 100 ns. In this way, the MCU can promptly turn off the laser in case of current generator malfunctioning or the Correlator board can provide a signal for fast-multiplexing of the light source.

Finally, simple optics focuses the light into the fiber to connect to the optical probe. An aspheric lens (A375TM-B, by Thorlabs Inc.) collimates the free-space laser's output and the light passes through an optical isolator (IO-3D-XXX-VLP series, by Thorlabs Inc.) to prevent laser damage due to back reflections. Then, a FiberPort collimator (PAF-X-15-PC-B, by Thorlabs Inc.) focuses the light into the fiber.

Taking advantage of the single mode fiber requirement for DCS detectors, we have built a ultra-light, low-profile, flexible optical probe which easily attaches to the head. To allow for a low probe profile, we use optical prisms in a rubber-like 3D printed probe head to optimize flexibility and contact with the tissue. Both fibers and prisms are inserted into the probe head, where they are glued with a two component, medical grade epoxy featuring very low viscosity and excellent optical-mechanical properties. The first 10 cm of the fibers are protected only by the black plastic coating (125 μm diameter) to maximize flexibility and minimize weight. After that the fibers are combined inside a protective jacket that facilitates handling and safeguards them from possible damage. The resulting probe is shown in FIG. 6. Each laser source is coupled to a 100 μm multi-mode fiber (numerical aperture=0.39) and attached to the same spot at the optical probe. At the probe end, the light is expanded to a larger area by bonding a 40° holographic diffuser between the source fibers and a 5.5 mm prism. The combination of the diffuser and the prism increases the angle of the incident light and minimizes the losses due to backscatter. The light is homogenously spread at the surface of the probe over a 5.5 mm diameter spot. This allows us to use higher optical power (up to 50 mW, resulting in 2.1 mW/mm²) while remaining within the ANSI Maximum Permissible Exposure (MPE) of skin to laser radiation: between 2.6 and 4 mW/mm² in the 760-850 nm range, as reported in the ANSI Standard Z136.1-1993 Table. Each detector is coupled to a 4.4 μm single-mode fiber to permit detection of single speckles. The detector fibers at the probe end are glued to 3 mm prisms at different distances from the source. Since the intensity of the detected light exponentially decreases with distance, at longer distances we use multiple detector fibers coupled to the same prism to improve the signal-to-noise ratio (SNR) (see, G. Dietsche, M. Ninck, C. Ortolf, J. Li, F. Jaillon, and T. Gisler, “Fiber-based multispeckle detection for time-resolved diffusing-wave spectroscopy: characterization and application to blood flow detection in deep tissue,” Applied Optics, vol. 46, no. 35, p. 8506 (2007)).

The light collected at the probe is sent to Single-Photon Avalanche Diode (SPAD) sensors able to detect light with single-photon sensitivity. Fast photon counting is required to measure the autocorrelation function. Key aspects to consider in the selection of these detectors are the photon detection efficiency (PDE) to maximize the detection of the collected light, the dark count rate (DCR) to optimize the signal-to-noise ratio, the afterpulsing probability and the linear relationship between input light and output count rate to minimize distortions when computing the DCS correlation curve.

The detectors employed (SPCM-850-14-FC, by Excelitas Technologies) have a PDE higher than 64% at 767 nm and higher than 54% at 852 nm. These detectors also have a low dead time (20 ns), resulting in an up to 40 Mcps count rate, allowing for a high SNR thanks to a low dark count rate (DCR), that is less than 100 cps. The afterpulsing probability is less than 3% and the detectors provide a linear relationship between input light and output count rate for up to 200 kcps, while at 1 Mcps there is a 2% distortion. Further characterization is necessary to determine the maximum conversion rate that guarantees a negligible distortion in the autocorrelation curve.

The last main block of this system is a custom-built correlator board shown in FIG. 4. The correlator is based on a field-programmable gate array (FPGA) device, that also hosts eight fast-comparators to translate the single-photon detector outputs to a proper pulse for the FPGA, and four analog channels to record analog traces. The FPGA time-tags each detected photon with an arrival time, by means of a counter locked to a 150 MHz clock, used as the time base. For maximum flexibility in the analysis, time-gating and autocorrelations are currently not implemented in the FPGA, but are instead performed by software (see, D. Wang, A. B. Parthasarathy, W. B. Baker, K. Gannon, V. Kavuri, T. Ko, S. Schenkel, Z. Li, Z. Li, M. T. Mullen, J. A. Detre, and A. G. Yodh, “Fast blood flow monitoring in deep tissues with real-time software correlators,” Biomedical Optics Express, vol. 7, no 3, pp. 776-797 (2016); and J, Dong, R. Bi, J. H. Ho, P. S. P. Thong, K. C. Soo and K. Lee “Diffuse correlation spectroscopy with a fast Fourier transform-based software autocorrelator,” Journal of Biomedical Optics, vol. 17, no. 9, p. 097004 (2012)) implementing a multi-tau scheme, after the data are transferred through a USB 3.0 interface. This allows us to select the integration time in post-processing, based on the measurement SNR. Because the acquisition rate is not limited by the software, we can quickly calculate autocorrelation functions (at a rate higher than 100 Hz), resulting in blood flow measurements with better than 10 ms resolution. This feature allows us to measure fast blood flow dynamics, which enable better physiological noise filtering and quantification of additional parameters (see, P. Farzam, J. Sutin, K.-C. Wu, B. Zimmermann, D. Tamborini, J. Dubb, D. A. Boas and M. A. Franceschini, “Fast diffuse correlation spectroscopy for non-invasive measurement of intracranial pressure,” Abstract of SPIE 10050, Clinical and Translational Neurophotonics, (2017)).

The Correlator board also handles the light source multiplexing and configures the laser currents to provide the same output power at each wavelength by setting the right driver current, since the output power versus current curve is different for each laser. Finally, the Correlator board by controlling the light multiplexing, also tags the photons at different wavelengths to guide the analysis.

A full characterization of the system was made by testing both the main components and the multi-distance multi-wavelength method with tissue-like phantoms experiments.

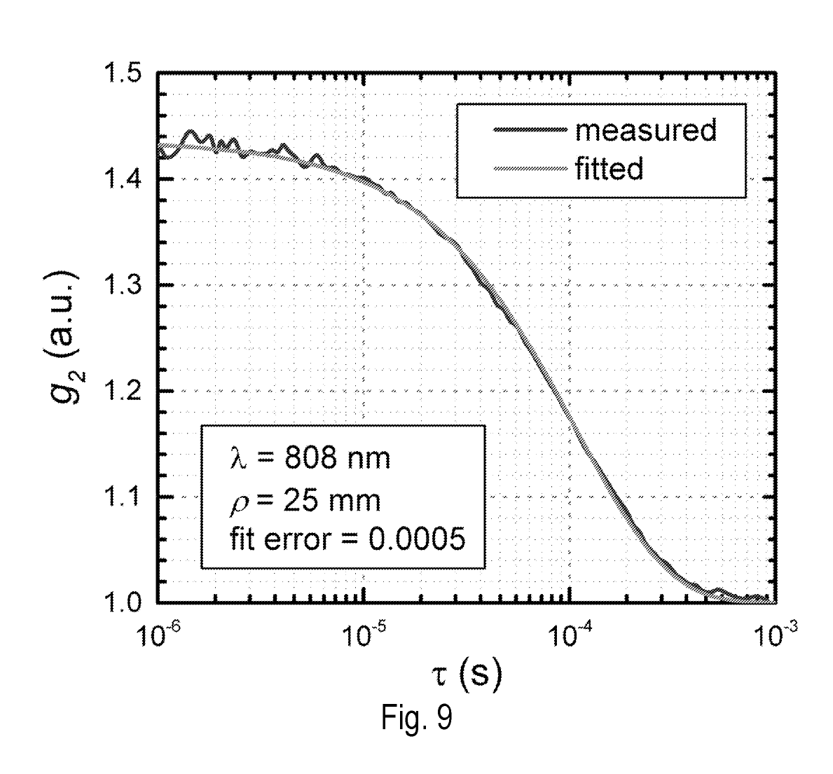

The detector performance has a significant impact on the quality of the DCS measurements. First, we verified the low detection noise and confirmed a DCR of 80-100 cps for all eight detectors. Then we tested the g₂ computation by performing measurements in a microsphere liquid phantom using our 808 nm DBR laser. We adjusted the light intensity to get a count rate of 200 kcps to operate the detector in the linear region. The resulting acquired normalized intensity autocorrelation function, g₂, and its post-processing fit are shown in FIG. 9. We verified that the estimated BFᵢ matched the microsphere proprieties with a low fitting error, computed as the squared norm of the fitting residual divided by the number of τ values. Finally, we characterized the potential impact of the non-linearity of the detector at high count rates (above 200 kcps) on the autocorrelation function to find the maximum count rate able to guarantee a negligible distortion in the g₂ curve.

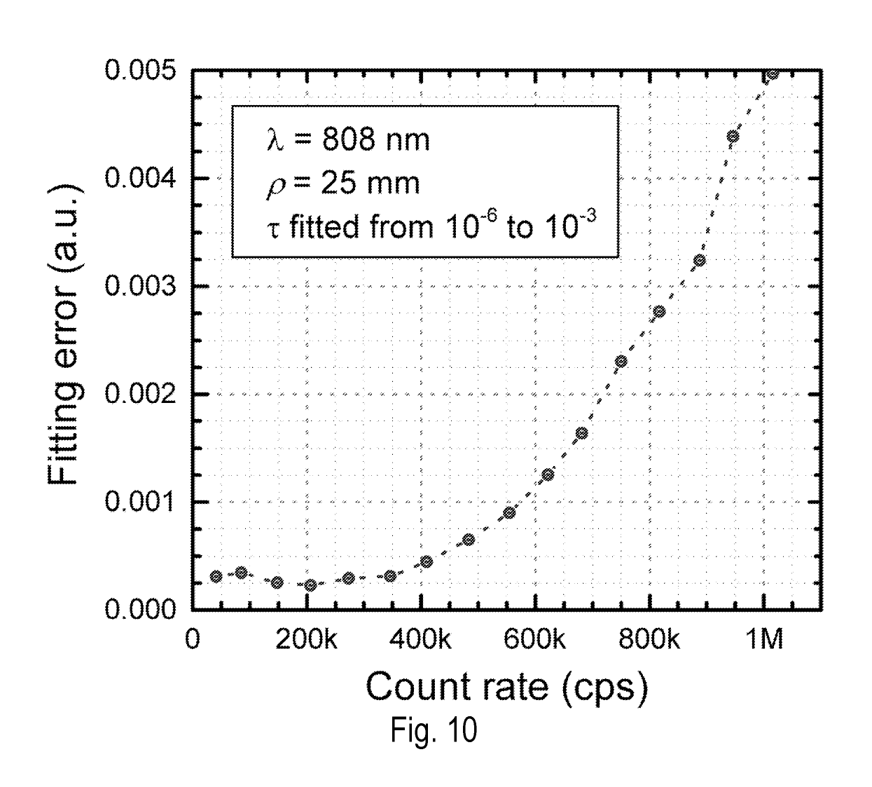

In DCS measurements, the signal-to-noise ratio increases with the number of photons used to compute a g₂ curve. Therefore, keeping a constant integration time, a higher detection count rate results in the autocorrelation function having higher SNR. The non-linearity of the detector at high count rates results in a distortion of the g₂ curve decay. Hence, we increased the light intensity, resulting in count rate between 50 kcps to 1 Mcps and computed the g₂ curves using the same amount of photons (5 million) to get the same SNR and evaluate only the distortion effect. In FIG. 10, we report the fitting error versus the detector count rate. The error, computed using the cost function (simplified version of eq. 10, with γ=0 and fitting only over τ), is constant for count rates up to 400 kcps and then rapidly increases an order of magnitude at 1 Mcps. To minimize distortion, the resulting optimal device operation is for a count rate no higher than 400 kcps.

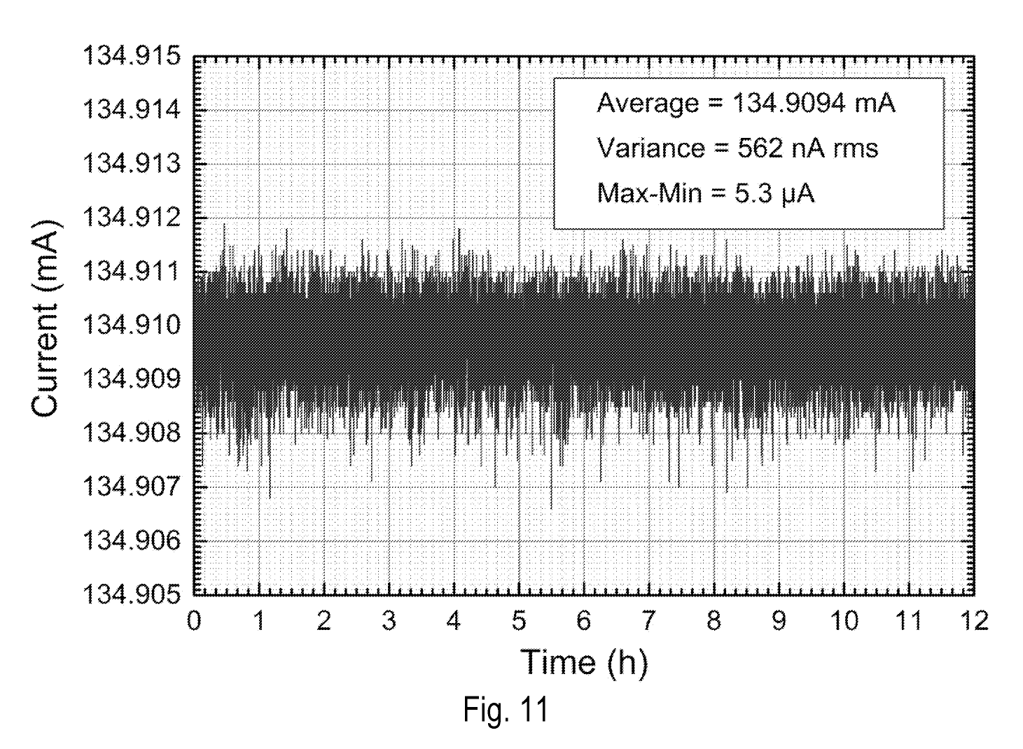

To successfully perform DCS measurements, stable long coherence length sources are required. We first verified the stability of our custom laser driver by measuring the current using a low-drift, sense resistor (Z Series Vishay Foil Resistors, by Vishay) as load and then acquiring its potential difference with a 6½ digit resolution digital multimeter (34401A, by Agilent). To achieve the maximum resolution of this multimeter we acquired the voltage across the resistor with a 2 sps rate for 12 hours, at room temperature (between 22-26° C.). The current is very stable, as shown in FIG. 11, without temperature drifting, thanks to the very low-drift electronic components employed. In fact, for a set current of 134.9 mA, we measured an average current of 134.9094 mA with a 562 nA rms standard deviation, resulting in less than 5 ppm variance.

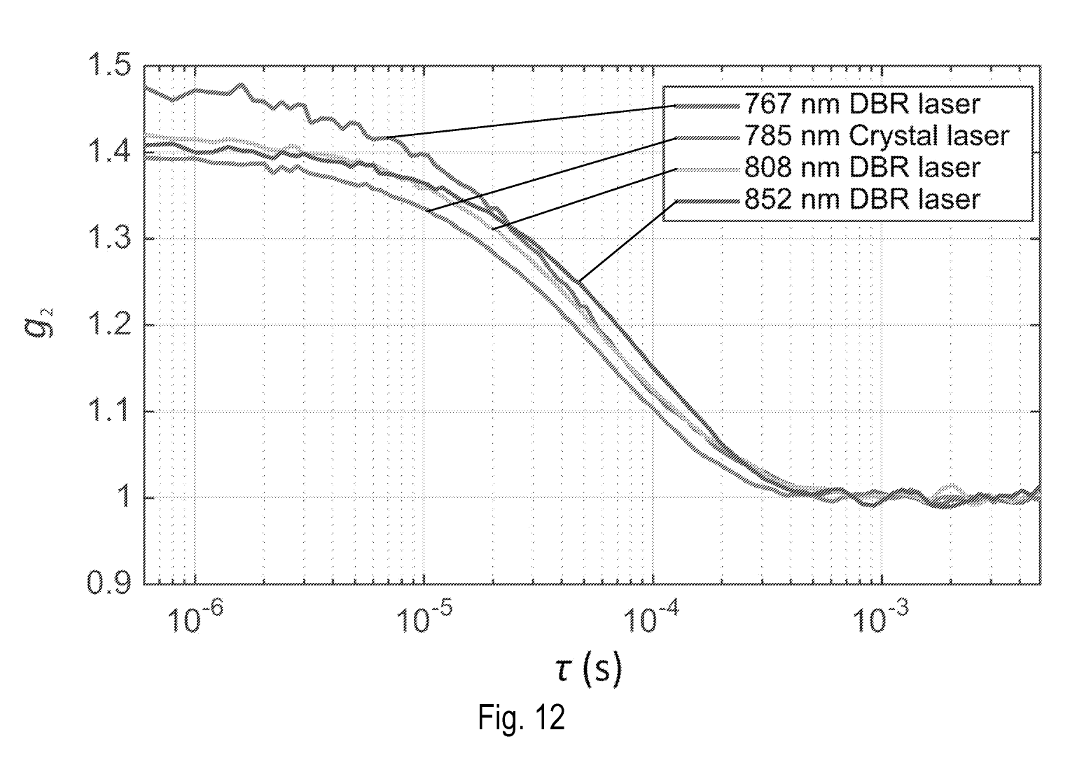

To test the coherence of the three DBR lasers we performed a DCS measurement on a liquid phantom, with a fixed source-detector distance of 25 mm, and comparing the three DBR lasers with results from a CrystaLaser laser at 785 nm (CL-2000 Diode Pumped Crystal, by CrystaLaser) typically used for DCS measurements. The autocorrelation functions (g₂) obtained with the four lasers are shown in FIG. 12, resulting in a β close to the theoretical maximum value of 0.5, for all lasers. The differences in decays are due to the different optical proprieties of the liquid phantom at the four wavelengths. This measurement proves the coherence length of the DBR lasers is sufficient for DCS measurements.

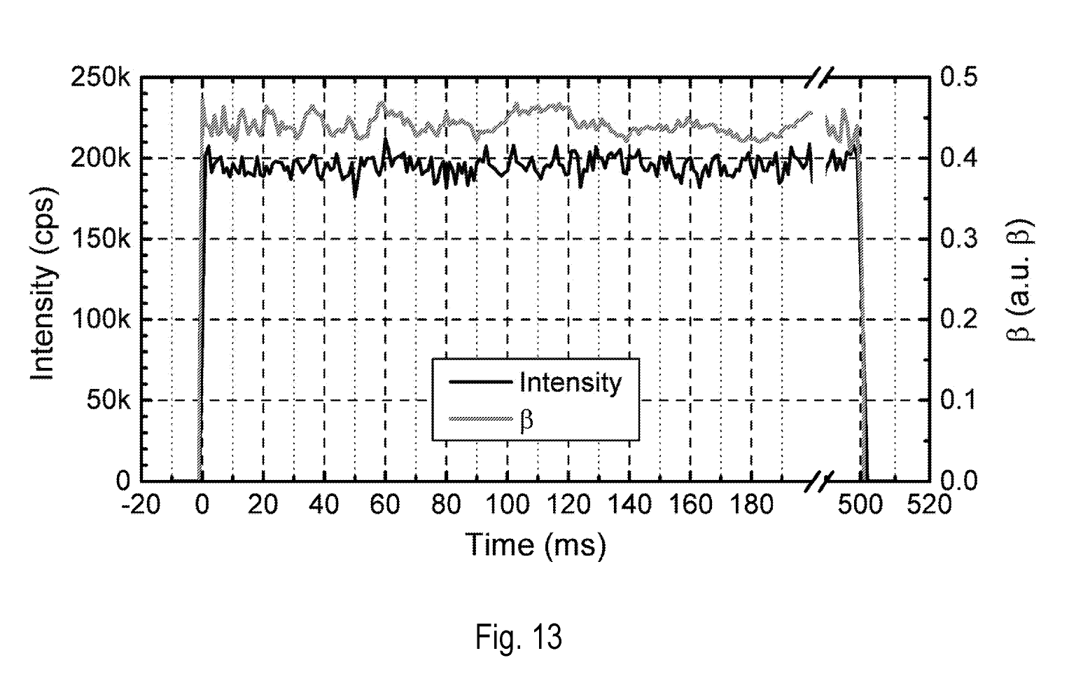

We measured the behavior of the DBR lasers when turning them on and off in rapid sequence to test whether the coherence length decreases with fast switching. We acquired data on a microsphere phantom and fed a 1 Hz trigger signal with 50% duty cycle to the 808 nm DBR laser to turn it on and off. We acquired the trigger signal through the analog channel to synchronize the g₂ curves with the laser on times. We computed g₂ curves and light intensity every 1 ms, from 20 ms before the detected rising edge to 520 ms after it. Since every 1 ms step has a limited number of photons (i.e., 200 photons for 200 kcps) resulting in a very noisy g₂ curve, we averaged the intensity and the g₂ curves over 900 periods. The coherence is evaluated as the β of the fitted autocorrelation curves. The behavior of the 808 nm laser is shown in FIG. 13, where both coherence and intensity are stable within 1 ms after the laser turn-on time, with a beta of 1.43±0.02 (mean value±standard deviation computed in the 900 periods) and a count rate of 196±13 kcps. Similar performances were observed with the other two lasers: beta of 1.41±0.02 with a 187±12 kcps count rate at 852 nm and a 1.47±0.03 beta with count rate of 242±17 kcps at 767 nm.

This shows a negligible system's warm-up time when the laser is switched on, allowing us to use a multiplexing time as short as tens of milliseconds and to rapidly acquire BFi at the three wavelengths in succession. We also evaluate the impact of the temporal multiplexing approach on the computation of the g₂ curves. Compared to a standard single-wavelength DCS system, in order to get BFᵢ with the same acquisition rate with this system we need to compute three g₂ curves (one per wavelength) in ⅓ of the time per wavelength. As described in C. Zhou, G. Yu, D. Furuya, J. H. Greenberg, A. G. Yodh and Turgut Durduran, “Diffuse optical correlation tomography of cerebral blood flow during cortical spreading depression in rat brain,” Optics Express, vol. 14, no. 3, pp. 1125-1144 (2006), the noise of the g₂ curves increase proportionally to \sqrt{t}, where t is the integration time. However, we could have excess noise as we also want to rapidly multiplex between wavelengths to make the measurements as nearly simultaneous as possible, but then integrate across multiplexed states to obtain a better SNR. The excess noise would arise from incomplete sampling of the correlation function if we multiplex on time scales approaching the decorrelation time scale. We experimentally verified that switching the lasers on-off at 25 ms intervals does not further increase the noise in g₂ with respect to CW operation. This was expected as 25 ms is much longer than the typical 10⁻⁵ to 10⁻⁴ decay time for our measured correlation functions.

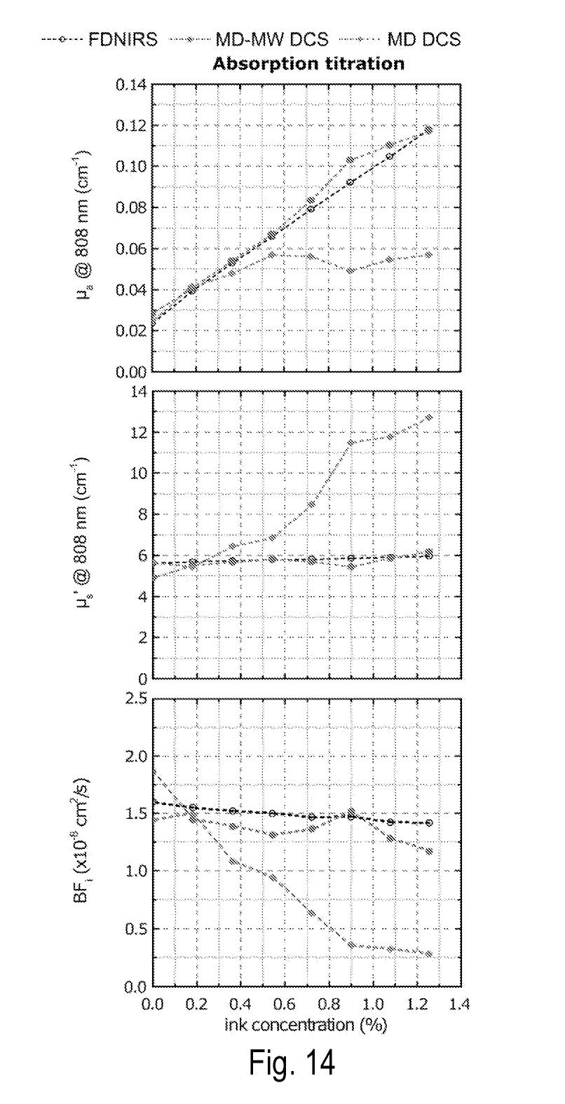

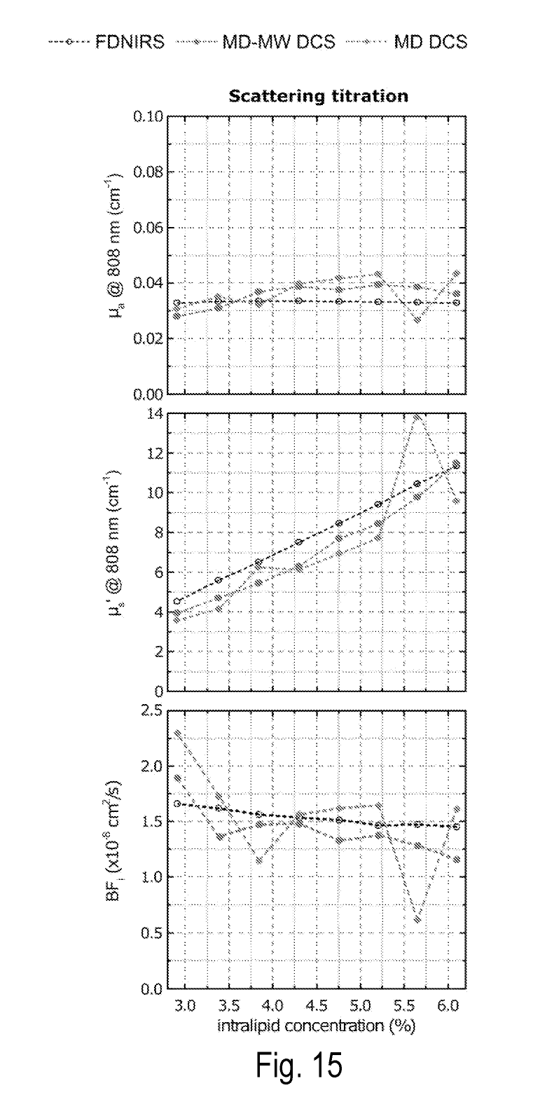

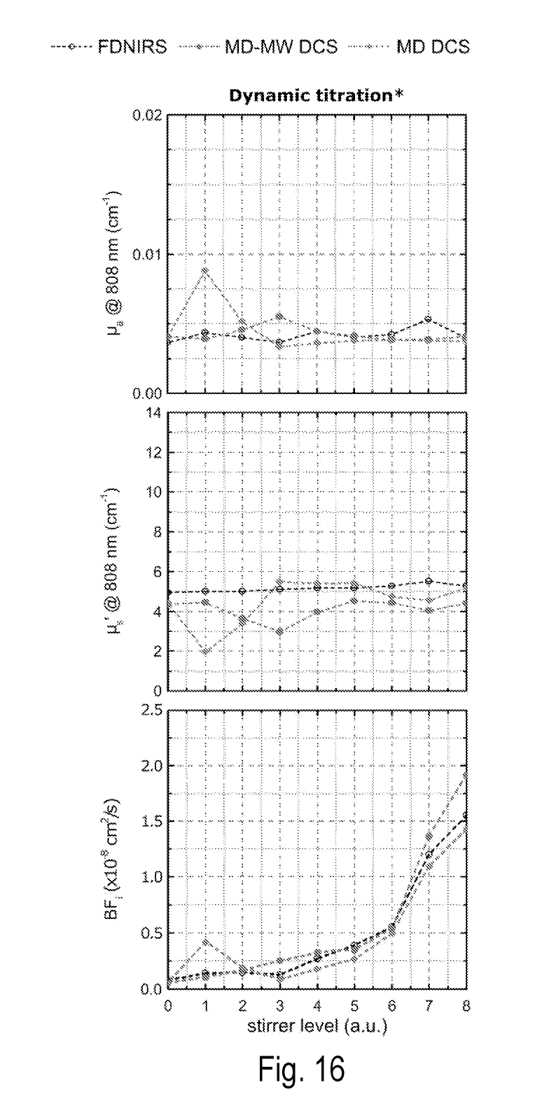

To experimentally demonstrate the robustness of the MD-MW DCS method we performed measurements on liquid phantoms while changing their optical proprieties and simultaneously fitting for a, b, ink concentration and BFᵢ. We compared the results of the MD-MW DCS method, with the results obtained using only the multi-distance information (MD DCS), and used the absorption and scattering measured with the FDNIRS system and the derived BFᵢ using these coefficients as the reference values (see, S. Kleiser, N. Nasseri, B. Andresen, G. Greisen and M. Wolf, “Comparison of tissue oximeters on a liquid phantom with adjustable optical properties,” Biomedical Optics Express, vol. 7, no. 8, pp. 2973-2992 (2016)). In FIGS. 14-16, we show the absorption (FIG. 14), scattering (FIG. 15), and dynamic (FIG. 16) titrations and from top to bottom we show the absorption coefficient, the reduced scattering coefficient and the mean square displacement of the solution as a function of titration level. For the absorption titration the computed absorption coefficients recovered with our MW-MD DCS approach linearly increase with the Ink concentration, and are in good agreement with the FDNIRS values with a maximum deviation of about 25%. The estimated reduced scattering coefficient and BFᵢ remain relatively constant during the absorption titration, revealing small cross-talk with changes in absorption. For the scattering titration the computed reduced scattering coefficients obtained with our method linearly increases with Intralipid concentration, in agreement with the FDNIRS results. The estimated absorption coefficient using the MD-MW DCS method remains relatively constant, while the BFᵢ shows a slight decrease with increased scattering on both FDNIRS and MD-MW DCS. For the dynamic titration the computed mean square displacement (BFᵢ) increases with the stirrer level and the recovered absorption and scattering coefficients remain relatively constant during the titration. For all titrations the computed parameters derived with the MD-MW DCS method are in good agreement with the parameters computed using the FDNIRS, with a maximum difference of about 25%. The only exception is during stirrer level 3 for which the absorption coefficient differs by about 33% and the reduced scattering coefficient differs by about 66% between the two modalities. By only using the multi-distance DCS information (red traces) we consistently obtained large deviations with the FDNIRS method, with differences in all computed parameters of up to 100-400%. These phantom measurements demonstrate that by adding the measures of intensity at multiple distances and DCS at multiple wavelengths, we add unique information that improves the ability to estimate μₐ, μₛ′, and BFᵢ, reaching a performance close to the state-of-the-art FDNIRS-DCS method.

We claim:

1. A multi-distance, multi-wavelength diffuse correlation spectroscopy (MD-MW DCS) system comprising:
one or more DCS light sources, the one or more DCS light sources configured to emit at least a first light having a first wavelength and a second light having a second wavelength, the one or more DCS light sources configured to transmit the first light and the second light into a target medium, the first wavelength and the second wavelength are different;
one or more DCS detectors, the one or more DCS detectors configured to receive at least a portion of the first light and at least a portion of the second light from the target medium, the DCS detector configured to generate a DCS detector signal in response to receiving the at least a portion of the first light and the at least a portion of the second light;
a memory storing one or more equations relating correlation to dynamics of scattering particles within the target medium; and
a processor coupled to the one or more DCS detectors and the memory, the processor configured to determine a dynamics of the target medium using the DCS detector signal and the one or more equations,
the one or more DCS light sources and the one or more DCS detectors configured to provide at least two different source-detector distances.

2. The system of claim 1, wherein the one or more DCS light sources are configured to transmit the first light and the second light into the target medium at a single transmission location and the one or more DCS detectors are two or more DCS detectors configured to receive the at least a portion of the first light and the at least a portion of the second light from the target medium at two different detection locations.

3. The system of claim 2, wherein a first DCS detector of the two or more DCS detectors is configured to receive light from the target medium at a first detector location positioned at a first source-detector distance relative to the single transmission location, and a second DCS detector of the two or more DCS detectors is configured to receive light from the target medium at a second detector location positioned a second source-detector distance relative to the single transmission location.

4. The system of claim 3, wherein a third DCS detector of the two or more DCS detectors is configured to receive light from the target medium at a third detector location positioned at a third source-detector distance relative to the single transmission location.

5. The system of claim 4, wherein a fourth DCS detector of the two or more DCS detectors is configured to receive light from the target medium at the third detector location.

6. The system of claim 4 or 5, wherein a fifth DCS detector of the two or more DCS detectors is configured to receive light from the target medium at a fourth detector location positioned at a fourth source-detector distance relative to the single transmission location.

7. The system of claim 6, wherein a sixth DCS detector of the two or more DCS detectors is configured to receive light from the target medium at the fourth detector location.

8. The system of claim 7, wherein a seventh DCS detector of the two or more DCS detectors is configured to receive light from the target medium at the fourth detector location.

9. The system of claim 8, wherein an eighth DCS detector of the two or more DCS detectors is configured to receive light from the target medium at the fourth detector location.

10. The system of claim 1, wherein the one or more DCS light sources are configured to transmit the first light and the second light into the target medium at two different transmission locations.

11. The system of claim 10, wherein the one or more DCS detectors are configured to receive the at least a portion of the first light and the at least a portion of the second light at a single detection location.

12. The system of claim 10, wherein the one or more DCS detectors are configured to receive the at least a portion of the first light and the at least a portion of the second light at two different detection locations.

13. The system of any one of the preceding claims, wherein the one or more DCS light sources are two or more DCS light sources, wherein a first DCS light source of the two or more DCS light sources is configured to emit the first light and a second DCS light source of the two or more DCS light sources is configured to emit the second light.

14. The system of claim 1, the one or more DCS light sources further configured to emit a third light having a third wavelength, the one or more DCS light sources configured to transmit the third light into the target medium, the first wavelength, the second wavelength, and the third wavelength are different, the one or more DCS detectors further configured to receive at least a portion of the third light from the target medium, the DCS detector configured to generate the DCS detector signal in response to receive the at least a portion of the first light, the at least a portion of the second light, and the at least a portion of the third light, the one or more DCS light sources and the one or more DCS detectors configured to provide at least three different source-detector distances.

15. The system of claim 14, wherein the one or more DCS light sources are configured to transmit the first light, the second light, and the third light into the target medium at a single transmission location and the one or more DCS detectors are three or more DCS detectors configured to receive the first light, the second light, and the third light from the target medium at three different detection locations.

16. The system of claim 15, wherein a first DCS detector of the three or more DCS detectors is configured to receive light from the target medium at a first detector location positioned at a first source-detector distance relative to the single transmission location, a second DCS detector of the three or more DCS detectors is configured to receive light from the target medium at a second detector location positioned at a second source-detector distance relative to the single transmission location, and a third DCS detector of the three or more DCS detectors is configured to receive light from the target medium at a third source-detector distance relative to the single transmission location.

17. The system of any one of claim 3 to 13 or 16, wherein the first source-detector distance is between 0.1 cm and 2.0 cm.

18. The system of any one of claim 3 to 13, 16 or 17, wherein the second source-detector distance is between 1.0 cm and 3.0 cm.

19. The system of any one of claims 4 to 13 or 16 to 18, wherein the third source-detector distance is between 1.0 cm and 5.0 cm.

20. The system of any one of claims 3 to 13 or 16 to 19, wherein the second source-detector distance is greater than the first source-detector distance.

21. The system of any one of claims 4 to 13 or 16 to 20, wherein the third source-detector distance is greater than the first source-detector distance and the second-source detector distance.

22. The system of any one of claims 16 to 21, wherein a fourth DCS detector of the three or more DCS detectors is configured to receive light from the target medium at a fourth detector location positioned at a fourth source-detector distance relative to the single transmission location.

23. The system of claim 22, wherein a fifth DCS detector of the three or more DCS detectors is configured to receive light from the target medium at the fourth detector location.

24. The system any one of claim 6 to 13 or 22 or 23, wherein the fourth source-detector distance is greater than the first source-detector distance, the second source-detector distance, and the third source-detector distance.

25. The system of any one of claims 6 to 13 or 22 to 24, wherein the fourth source-detector distance is between 1.0 cm and 6.0 cm.

26. The system of any one of claims 22 to 25, wherein a sixth DCS detector of the three or more DCS detectors is configured to receive light from the target medium at a fifth detector location positioned at a fifth source-detector distance relative to the single transmission location.

27. The system of claim 26, wherein a seventh DCS detector of the three or more DCS detectors is configured to receive light from the target medium at the fifth detector location.

28. The system of claim 27, wherein an eighth DCS detector of the three or more DCS detectors is configured to receive light from the target medium at the fifth detector location.

29. The system of any one of claims 26 to 28, wherein the fifth source-detector distance is greater than the first source-detector distance, the second source-detector distance, the third source-detector distance, and the fourth source-detector distance.

30. The system of any one of claims 26 to 29, wherein the fifth source-detector distance is between 1.0 cm and 6.0 cm.

31. The system of claim 1, wherein the one or more DCS light sources are configured to transmit the first light, the second light, and the third light into the target medium at three different transmission locations.

32. The system of claim 31, wherein the one or more DCS detectors are configured to receive the at least a portion of the first light, the at least a portion of the second light, and the at least a portion of the third light from the target medium at a single detection location.

33. The system of claim 31, wherein the one or more DCS detectors are configured to receive the first light, the second light, and the third light from the target medium at three different detection locations.

34. The system of any one of claims 14 to 33, wherein the one or more DCS light sources are three or more DCS light sources, wherein a first DCS light source of the three or more DCS light sources is configured to emit the first light, a second DCS light source of the three or more DCS light sources is configured to emit the second light, and a third DCS light source of the three or more DCS light sources is configured to emit the third light.

35. The system of any one of the preceding claims, wherein the one or more DCS light sources includes a diode laser, a solid-state laser, a fiber laser, a vertical cavity surface-emitting laser (VCSEL), a DBR laser, a Fabry-Perot laser, a ridge laser, a ridge waveguide laser, a tapered laser, or a combination thereof.

36. The system of any one of the preceding claims, wherein the one or more DCS light sources includes a diode laser, a solid-state laser, a fiber laser, of a combination thereof.

37. The system of any one of the preceding claims, wherein one or more DCS light sources are configured to emit light at a wavelength of between 400 nm and 1800 nm.

38. The system of any one of the preceding claims, wherein the one or more DCS light sources is configured to emit light at an average power of between 10 μW and 10 W.

39. The system of any one of the preceding claims, the system further comprising a light source driver coupled to the computer and the one or more DCS light sources.

40. The system of claim 39, wherein the light source driver is configured to control the one or more light sources to multiplex the first and second light.

41. The system of any one of the preceding claims, the system further comprising an additional light source.

42. The system of claim 41, wherein the additional light source is a near infrared spectroscopy light source.

43. The system of any one of the preceding claims, the system further comprising an additional detector.

44. The system of claim 43, wherein the additional detector is a near infrared spectroscopy detector.

45. The system of any one of the preceding claims, wherein the one or more DCS detectors includes a detector selected from the group consisting of a single-photon avalanche photodiode detector, a photomultiplier tube, a Si, Ge, InGaAs, PbS, PbSe or HgCdTe photodiode or PIN photodiode, phototransistors, MSM photodetectors, CCD and CMOS detector arrays, LCD, silicon photomultipliers, multi-pixel-photon-counters, and combinations thereof.

46. The system of claim 45, wherein the one or more DCS detectors are one or more single-photon avalanche photodiode detectors.

47. The system of any one of the preceding claims, wherein the DCS detector signal is an analog signal, a digital signal, a photon-counting signal, or a combination thereof.

48. The system of any one of the preceding claims, the system further comprising one or more waveguides configured to couple the one or more DCS light sources to the target medium or configured to couple the target medium to the one or more DCS detectors.

49. The system of any one of the preceding claims, the system further comprising one or more lenses configured to couple the one or more DCS light sources to the target medium or configured to couple the target medium to the one or more DCS detectors.

50. The system of any one of the preceding claims, wherein the system is contained in one or more handheld units.

51. The system of any one of the preceding claims, wherein the one or more DCS detectors are configured to collect light from one speckle.

52. The system of any one of the preceding claims, wherein the first wavelength and the second wavelength are separated from one another by between 20 nm and 500 nm.

53. A method for making a multiple distance, multiple wavelength diffuse correlation spectroscopy (MD-MW DCS) measurement of scattering particle dynamics within a target medium, the method comprising:
a) coupling one or more DCS light sources and one or more DCS detectors to the target medium to provide at least two different source-detector distances, the one or more DCS light sources configured to emit at least a first light having a first wavelength and a second light having a second wavelength, the first wavelength and the second wavelength are different;
b) transmitting the first light and the second light into the target medium;
c) receiving at least a portion of the first light and at least a portion of the second light at the one or more DCS light detectors at both of the at least two different source-detector distances, thereby generating a DCS detector signal including photon arrival time information, wavelength information, and source-detector distance information;
d) determining, using a processor and the DCS detector signal, a decay of an autocorrelation function over distance for at least the first wavelength and the second wavelength;
e) determining, using the processor, the decay of the autocorrelation function over distance, and one or more equations relating the decay of the autocorrelation function over distance to optical properties and dynamics of the target medium, the dynamics of the target medium; and
f) generating a report including the dynamics of the target medium.

54. A method comprising:
a) coupling one or more DCS light sources and one or more DCS detectors to the target medium, the one or more DCS light sources configured to emit at least a first light having a first wavelength and a second light having a second wavelength, the first wavelength and the second wavelength are different, the one or more DCS light sources and the one or more DCS detectors are configured to provide at least two different source-detector distances;
b) transmitting the first light and the second light into the target medium;
c) receiving at least a portion of the first light and at least a portion of the second light at the one or more DCS light detectors at each of the two different source-detector distances, thereby generating a DCS detector signal including light intensity, autocorrelation, wavelength information, and source-detector distance information;
d) determining, using (1) a processor, (2) the DCS detector signal, and (3) a global fitting method, (i) an absorption coefficient (μₐ), (ii) a reduced scattering coefficient (μₛ′), and (iii) a blood flow index (BFi); and
e) generating a report including the absorption coefficient, the reduced scattering coefficient, or the blood flow index.

55. The method of claim 53 or 54, wherein step b) includes multiplexing the first light and the second light prior to the transmitting.

56. The method of any one of claims 53 to 55, wherein the one or more DCS light sources includes a first light source configured to emit the first light and a second light source configured to emit the second light.

57. The method of any one of claims 53 to 56, wherein the transmitting of step b) includes transmitting the first light and the second light into the target medium at a single transmission location.

58. The method of any one of claims 55 to 57, wherein the DCS detector signal thereby generated by the receiving of step c) is an analog signal, a digital signal, a photon-counting signal, or a combination thereof.

59. The method of claim 58, wherein the DCS detector signal thereby generated by the receiving of step c) is the analog signal.

60. The method of claim 58, wherein the DCS detector signal thereby generated by the receiving of step c) is the digital signal.

61. The method of claim 58, wherein the DCS detector signal thereby generated by the receiving of step c) is the photon-counting signal.

62. The method of any one of claims 53 to 61, wherein the first light and the second light each has a wavelength of between 400 nm and 1800 nm.

63. The method of any one of claims 53 to 62, wherein the determining of step f) includes fitting data.

64. The method of claim 63, wherein the fitting data is achieved using a global fitting method.

65. The method of any one of claims 53 to 64, wherein the dynamics of the target medium include a blood flow index.

66. The method of any one of claims 53 to 65, wherein the dynamics of the target medium include a fluid flow within the target medium.

67. The method of claim 66, wherein the target medium is tissue and the fluid flow within the target medium is a blood flow within the tissue.

68. The method of claim 67, wherein the blood flow is pulsatile blood flow.

69. The method of claim 68, the method further comprising physiologically noise filtering the dynamics using the pulsatile blood flow.

70. The method of claim 68 or 69, the method further comprising quantifying cerebrovascular reactivity or intracranial pressure.

71. The method of any one of claims 53 to 70, the coupling of step a) providing at least three different source-detector distances, the receiving of step c) being at each of the at least three different source-detector distances.

72. The method of any one of claims 53 to 71, the one or more DCS light sources further configured to emit a third light having a third wavelength, the first wavelength, the second wavelength, and the third wavelength are different, the transmitting of step b) including transmitting the third light into the target medium, the receiving of step c) including receiving at least a portion of the third light at the one or more DCS light detectors at both of the at least two different source-detector distances, thereby generating the DCS detector signal, and the determining of step d) using a decay of the autocorrelation function over distance for the third wavelength.
